(19) United States
(12) Patent Application Publication
(10) Pub. No.: US 2018/0147198 A1
(43) Pub. Date: May 31, 2018

(54) COMBINATIONS OF PFKFB3 INHIBITORS AND IMMUNE CHECKPOINT INHIBITORS TO TREAT CANCER

(71) Applicant: UNIVERSITY OF LOUISVILLE RESEARCH FOUNDATION, INC., Louisville, KY (US)

(72) Inventors: Jason CHESNEY, Louisville, KY (US); Sucheta TELANG, Louisville, KY (US); Kavitha YADDANAPUDI, Louisville, KY (US)

(73) Assignee: UNIVERSITY OF LOUISVILLE RESEARCH FOUNDATION, INC., Louisville, KY (US)

(21) Appl. No.: 15/576,877

(22) PCT Filed: May 27, 2016
(86) PCT No.: PCT/US16/34590

§ 371 Date: Nov. 27, 2017

Related U.S. Application Data
(60) Provisional application No. 62/167,403, filed on May 28, 2015.

(51) Int. Cl. A61K 31/4709; A61P 35/00; A61K 39/395; C07K 16/28; A61K 9/00

(52) U.S. Cl. CPC A61K 31/4709; A61P 35/00; A61K 2039/505; C07K 16/2818; A61K 9/0019; A61K 39/39541

(57) ABSTRACT

Provided herein is a method of treating cancer and stimulating anti-tumor immunity in a subject in need thereof, the method including administering to the subject a synergistic, therapeutically effective amount of a PFKFB3 inhibitor, such as PFK-158, in combination with an immune checkpoint inhibitor. Also provided is a method of synergistically increasing activity of an immune checkpoint inhibitor, the method including administering to a subject in need thereof a combination therapy including PFK-158 and the immune checkpoint inhibitor. A pharmaceutical composition including PFK-158, at least one immune checkpoint inhibitor; and at least one pharmaceutically-acceptable carrier is also provided.

### CROSS REFERENCE TO RELATED APPLICATIONS

This application claims the benefit of U.S. Provisional Application No. 62/167,403, filed May 28, 2015, which is incorporated herein by reference in its entirety.

### FIELD OF THE INVENTION

The present disclosure relates to the field of cancer therapy. Specifically, the present disclosure relates to methods of treating cancer and activating anti-tumor immunity by administering (E)-1-(pyridyn-4-yl)-3-(7-(trifluoromethyl)quinolin-2-yl)-prop-2-en-1-one (PFK-158) to a subject in need thereof, particularly in combination with an immune checkpoint inhibitor.

### BACKGROUND

T cell activation is associated with a rapid increase in intracellular fructose-2,6-bisphosphate (F2,6BP), an allosteric activator of the glycolytic enzyme, 6-phosphofructo-1-kinase. The steady state concentration of F2,6BP in T cells is dependent on the expression of the bifunctional 6-phosphofructo-2-kinase/fructose-2,6-bisphosphatases (PFKFB1-4). Of the PFKFB family of enzymes, PFKFB3 has the highest kinase:bisphosphatase ratio and has been demonstrated to be required for T cell proliferation. A recent study showed that purified human CD3+ T cells express PFKFB2, PFKFB3, PFKFB4 and TIGAR, and that anti-CD3/anti-CD28 conjugated microbeads stimulated a >20-fold increase in F2,6BP with a coincident increase in protein expression of the PFKFB3 family member. Telang, et al, Small molecule inhibition of 6-phosphofructo-2-kinase suppresses t cell activation, J. Transl. Med. 10:95 (2012). The study further showed that exposure to the small molecule antagonist of PFKFB3, 3-(3-pyridinyl)-1-(4-pyridinyl)-2-propen-1-one (3PO) (1-10 μM), markedly attenuated the stimulation of F2,6BP synthesis, 2-[1-14C]-deoxy-D-glucose uptake, lactate secretion, TNF-α secretion and T cell aggregation and proliferation. See Telang, et al. The in vivo effect of 3PO on the development of delayed type hypersensitivity to methylated BSA and on imiquimod-induced psoriasis in mice was examined and showed that 3PO suppressed the development of both T cell-dependent models of immunity in vivo. See Telang, et al. These data demonstrated that inhibition of the PFKFB3 kinase activity attenuates the activation of T cells in vitro and suppresses T cell dependent immunity in vivo and suggested that small molecule antagonists of PFKFB3 may prove effective as T cell immunosuppressive agents.

Derivatives of 3PO have since been developed and one, (E)-1-(pyridyn-4-yl)-3-(7-(trifluoromethyl)quinolin-2-yl)-prop-2-en-1-one (PFK-158), is now under study in a multi-center phase 1 trial. PFK-158 has the following structure:

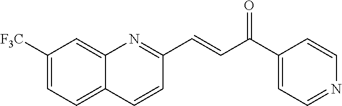 [SMILES: O=C(/C=C/c1ccc2ccc(C(F)(F)F)cc2n1)c1ccncc1]

Given the potential immunosuppressive activity of PFKFB3 inhibitors, there has been and continues to be concern that the anti-tumor activity of PFK-158 would be attenuated by concurrent immunosuppression. However, the dose escalation portion of the phase 1 trial has already shown several patients experiencing stable disease up to six months. Although the in vitro effective dose of PFK-158 to induce cytotoxicity in cancer cells was greater than expected in the early cohorts of the phase 1 trial, a clear clinical response in an ocular melanoma patient was observed. It was thus postulated that an effect of PFK-158 on the non-tumor host cells may be contributing to the clinical activity. The immunological effects of PFK-158 in a pre-clinical mouse model of melanoma (B16-F10) were further studied in order to assess the concerns related to immunosuppression.

Although immunosuppressive cells such as Th17 cells and γδT17 cells are well established to attenuate the induction of tumor immunity in mouse and human studies, pharmacological targeting of these cells has proven difficult. Th17 cells and γδT17 cells are attractive targets since they produce IL-17, which not only suppresses tumor immunity via promotion of MDSCs, but also supports angiogenesis.

The development of immune checkpoint inhibitors (ICIs) has resulted in a marked reduction in lung cancer- and melanoma-related deaths. Immune checkpoint inhibitors indirectly treat cancer by treating the immune system, acting as the off-switch for T cells. ICIs that unblock an existing immune response or unblock the initiation of an immune response have shown promise in some subjects. Certain ICIs, such as cytotoxic T-lymphocyte-associated antigen 4 (CTLA-4) and programmed death 1 T cell receptor (PD-1), have attracted attention in recent years as potential cancer targets.

CTLA-4, PD-1, and their ligands are members of the CD28-B7 family of co-signaling molecules that play important roles throughout all stages of T-cell and other cell functions. The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and essentially switches it off or inhibits it. Cancer cells take advantage of this system by driving high levels of expression of PD-L1 on their surface. This allows cancer cells to gain control of the PD-1 pathway and switch off T cells expressing PD-1 that may enter the tumor microenvironment, thus suppressing the anticancer immune response.

The immunotherapy ipilimumab, a monoclonal antibody that targets CTLA-4 on the surface of T cells, has been approved for the treatment of melanoma. Various new targeted immunotherapies aimed at the programmed death-1 (PD-1) T-cell receptor or its ligands (PD-L1 or PD-L2) may also prove to be effective. Additional checkpoint targets may also prove to be effective, such as TIM-3, LAG-3, various B-7 ligands, CHK1 and CHK2 kinases, BTLA, A2aR, and others.

Various immune checkpoint inhibitors are currently in clinical study. There is a need to develop improved methods of treating cancer and stimulating the effectiveness of immune checkpoint inhibitors.

### SUMMARY OF THE INVENTION

Accordingly, provided herein are methods and compositions for treating cancer, stimulating, increasing, or modulating anti-tumor immunity and/or synergistically stimulating, increasing, or modulating the activity of an immune checkpoint inhibitor.

In one embodiment, a method of treating cancer is provided, comprising administering to a subject in need thereof a synergistic, therapeutically effective amount of (1) (E)-1-(pyridyn-4-yl)-3-(7-(trifluoromethyl)quinolin-2-yl)-prop-2-en-1-one (PFK-158); and (2) an immune checkpoint inhibitor.

In another embodiment, a method of stimulating anti-tumor immunity is provided, comprising administering to a subject in need thereof a therapeutically effective amount of PFK-158.

In another embodiment, a method of synergistically increasing activity of an immune checkpoint inhibitor is provided, comprising administering to a subject in need thereof a synergistic, therapeutically effective amount of (1) (E)-1-(pyridyn-4-yl)-3-(7-(trifluoromethyl)quinolin-2-yl)-prop-2-en-1-one (PFK-158) and (2) the immune checkpoint inhibitor.

In another embodiment, a pharmaceutical composition is provided, comprising a therapeutically effective amount of PFK-158; a therapeutically effective amount of at least one immune checkpoint inhibitor; and at least one pharmaceutically-acceptable carrier.

These and other objects, features, embodiments, and advantages will become apparent to those of ordinary skill in the art from a reading of the following detailed description and the appended claims.

### BRIEF DESCRIPTION OF THE DRAWINGS

FIG. 1 shows flow cytometric analyses of cells indicating that PFKFB3 inhibition with PFK-158 depletes immunosuppressive splenic Th17 and γδT17 cells in melanoma-bearing mice. Control spleen CD4+CD3+IL-17+(A); Control spleen γδT+IL17+(B); +158 spleen CD4+CD3+IL-17+(C); +158 spleen γδT+IL17+(D).

FIG. 2 shows flow cytometric analyses of cells indicating that PFKFB3 inhibition with PFK-158 depletes immunosuppressive splenic and intratumoral Th17 cells and γδT17 cells in melanoma-bearing mice. Control spleen RORγT (A); Control tumor RORγT (B); Control spleen γδ+RORγ T+(C); Control tumor γ+RORγ T+(D); +158 spleen RORγT (E); +158 tumor RORγT (F); +158 spleen γδ+RORγ T+(G); +158 tumor γδ+RORγ T+(H).

FIG. 3 shows flow cytometric analyses of cells indicating that PFKFB3 inhibition with PFK-158 depletes immunosuppressive intratumoral CD4+ and CD8+ Treg cells in melanoma-bearing mice. Control CD4+FOXP3+(A); Control CD8+FOXP3+(B); +158 tumor CD4+FOXP3+(C); +158 tumor CD8+FOXP3+(D).

FIG. 4 shows flow cytometric analyses of cells indicating that PFKFB3 inhibition with PFK-158 depletes immunosuppressive MDSCs in melanoma-bearing mice. Control (A) and +PFK-158 (B).

FIG. 5 shows flow cytometric analyses of cells indicating that PFKFB3 inhibition with PFK-158 increases intratumoral CD4+ and CD8+ T cells. (A) shows results for CD4+T at 3 days, comparing control (left) and tumor treated with PFK-158 (right). (B) shows results for CD8+ T cells at 3 days, comparing control (left) and tumor treated with PFK-158 (right). (C) shows results for CD8+ T cells increased in tumors, comparing isotype control (left), CD3+CD8+ control tumor (center), and PFK-FB3 treated tumor (right).

FIG. 6 shows flow cytometric analyses of cells indicating that PFKFB3 inhibition with PFK-158 increases intratumoral B cells. Control tumor CD3-CD19+(A); +158 tumor CD3-CD19+(B).

FIG. 7 is a graph showing PFKFB3 inhibition with PFK-158 improves the immunotherapy anti-CTLA-4 antibody in melanoma-bearing mice, comparing treatment with vehicle, anti-CTLA-4 antibody, PFK-158, and anti-CTLA-4 antibody+PFK-158.

FIG. 8 shows flow cytometric analyses of cells indicating that PFKFB3 inhibition with PFK-158 depletes peripheral blood Th17 cells in a human subject. Peripheral blood mononuclear cells were collected on days 1, 8, 15, 22 and 62 and flow cytometry was conducted for a multitude of immunosuppressive cells and activated T cells. Immunosuppressive cells in the peripheral blood was quantified for a healthy donor (A); at baseline (day 1; C1D1) (B); and after PFK-158 administration (days 8 (C1D8) (C), 15 (C1D15) (D), 22 (C1D22) (E) and 62 (C3D5) (F) were examined and reductions were observed in Th17 cells.

FIG. 9 shows flow cytometric analyses of cells indicating that PFKFB3 inhibition with PFK-158 depletes peripheral blood γδT17 cells in a human subject. Peripheral blood mononuclear cells were collected on days 1, 8, 15, 22 and 62 and flow cytometry was conducted for a multitude of immunosuppressive cells and activated T cells. Immunosuppressive cells in the peripheral blood was quantified for a healthy donor (A); at baseline (day 1; C1D1) (B); and after PFK-158 administration days 8 (C1D8) (C), 15 (C1D15) (D), 22 (C1D22) (E) and 62 (C3D5) (F) were examined and reductions were observed in γδT17 cells.

FIG. 10 shows flow cytometric analyses of cells indicating that PFKFB3 inhibition with PFK-158 depletes peripheral blood Treg cells in a human subject. Healthy donor CD4 (left panel) and CD127 (right panel) (A); C1D1 CD4 (left panel) and CD127 (right panel) (B); C1D8 CD4 (left panel) and CD127 (right panel) (C); C1D15 CD4 (left panel) and CD127 (right panel) (D); C1D22 CD4 (left panel) and CD127 (right panel) (E); and C3D5 CD4 (left panel) and CD127 (right panel) (F).

FIG. 11 shows flow cytometric analyses of cells indicating that PFKFB3 inhibition with PFK-158 increases peripheral blood activated CD4+ T cells in a human subject. CD4 and CD69 are evaluated for healthy donor (A); day 1, C1D1 (B); day 8, C1D8 C); day 15, C1D15 (D); day 22, C1D22 (E); and day 62, C3D5 (F).

FIG. 12 shows flow cytometric analyses of cells indicating that PFKFB3 inhibition with PFK-158 increases peripheral blood activated CD8+ T cells in a human subject. CD8 and CD69 are evaluated for healthy donor (A); day 1, C1D1 (B); day 8, C1D8 C); day 15, C1D15 (D); day 22, C1D22 (E); and day 62, C3D5 (F).

FIG. 13 shows flow cytometric analyses of cells indicating that PFKFB3 inhibition with PFK-158 increases peripheral blood interferon-alpha and tumor necrosis factor-alpha positive CD8+ T Cells in a human subject for healthy donor (A); day 1, C1D1 (B); day 8, C1D8 (C); day 15, C1D15 (D); day 22, C1D22 (E); and day 62, C3D5 (F). For each of (A), (B), (C), (D), (E), and (F), the left panel shows results for CD8+ T cells, the center panel shows results for CD8+IFNγ+ cells, and the right panel shows results for CD8+TNFα+ cells.

FIG. 14 shows flow cytometric analyses of cells indicating that PFKFB3 inhibition with PFK-158 increases peripheral blood cancer-reactive CD8+ T cells for day 1, C1D1 (A); day 8, C1D8 (B); day 15, C1D15 (C); day 22, C1D22 (D); and day 62, C3D5 (E). For each of parts (A), (B), (C), (D), (E), and (F), the left panel shows results for CD8+ T cells, the center panel shows results for CD8+CD57+ cells, and the right panel shows results for CD27+CD27− cells.

FIG. 15 shows flow cytometric analyses of cells and graphs demonstrating PFKFB3 inhibition with PFK-158 decreases PD-1 expression on peripheral blood cancer-reactive CD8+ T cells in a human subject. C1D1 (A); C1D8 (B); C1D15 (C); C1D22 (D); and C3D5 (E).

FIG. 16 shows selective inhibition of PFKFB3 decreases tumor-infiltrating Th17 and γδT17 cells in vivo. Percentages of γδT17 cells were decreased (A) (left panel vehicle, center panel+PFK-158, right panel comparison), and (B) Th17 cells were decreased (left panel vehicle, center panel+PFK-158, right panel comparison), whereas percentages of CD8+IFNγ+ T cells were increased in the tumors after PFK-158 administration (C) (left panel vehicle, center panel+PFK-158, right panel comparison).

FIG. 17 shows analysis of Th17 cells and human naïve Vγ9Vδ2 T cells for PFKFB2-4 mRNA (A) and (B); F2,6BP and PFKFB3 (C) and (D). CD4+ T cells and Vγ9Vδ2 T cells were exposed to vehicle or PFK-158 at differing concentrations and IL-17 and production was quantified by ELISA (E) and (F).

FIG. 18 shows regression of hepatic metastasis in ocular melanoma patient receiving PFK-158 for baseline (A); +2 cycles (B); +4 cycles (C); and +6 cycles (D). Depicted is a CT imaging slice of a hepatic metastasis that became necrotic after 2 cycles and then regressed.

FIG. 19 shows PFK-158 depletes Th17 cells and increases CD8+/IFNγ+ T cells in a breast cancer patient. After administration of PFK-158 for 4 cycles, bony metastases were deemed to be stable (left two panels) and several liver metastases became necrotic (right panel).

FIG. 20 shows the effect of PFKFB3 inhibition on peripheral blood CD8+CD57+CD27-CD28− T cells. A breast cancer patient was administered PFK-158 for 1 cycle and PBMCs were collected at baseline (row 1), day 8 (row 2), day 15 (row 3), and day 22 (row 4) of treatment, and then analyzed for CD8+CD57+CD27-CD28− T cells and PD-1 expression.

FIG. 21 shows inhibition of PFKFB3 increases the activity of anti-CTLA-4 antibody in vivo. B16-F10 tumor bearing C57BL/6 mice were administered vehicle, anti-CTLA-4 antibody (0.1 mg i.p. every third day×3), PFK-158 (0.06 mg·gm i.p.×3 days and then every other day), and a combination of anti-CTLA-4 antibody and PFK-158.

FIG. 22 shows the effect of PFK-158 on multiple circulating immune cells, including CD3+CD4+IL17+ cells, CD3+gdT+IL-17+ cells, Treg cells (CD4+CD25+CD127Low), and M-MDSCs (CD14+CD11b+HLA-DRLow/−CD33+) (A); CD4+CD69+ T cells (B); and CD8+CD69+, CD8+IFN-γ+, CD8+CD27-CD28-CD57+, and CD8+CD137+ T cells (C).

FIG. 23 shows the effect of PFKFB3 inhibition by PFK-158 on peripheral blood Th17 cells and tumor antigen-reactive CD137+/CD8+ T cells. Four patients having metastatic cancer, including breast cancer (row 1), renal cell carcinoma (row 2), ovarian cancer (row 3), and esophageal cancer (row 4), were administered PFK-158 for 2 weeks and PBMCs were collected at baseline (columns 1 and 3) and day 15 (columns 2 and 4) and analyzed by flow cytometry for CD3+cd4+IL-17+ cells (columns 1 and 2) and CD8+CD137+ T cells (columns 3 and 4).

FIG. 24 shows genomic deletion of Pfkfb3 depletes tumor-infiltrating Th17 and γδT17 cells and the growth of Pfkfb3 wild-type B16 tumors. Tam-β-ActinCre:Pfkfb3fl/fl mice at 16 weeks of age were injected with corn oil (Pfkfb3 WT) or tamoxifen (Pfkfb3 KO+TAM, 200 mg/kg×5 days, i.p.) and 2 days later were implanted with 1×10⁵ B16F10 melanoma cells in the flank. 3 mice were euthanized after 4 days for analysis of tumor-infiltrating CD4+/IL-17+(A), γdT+/IL-17+(B), CD4+/ROR-γt+(C), γdT+/ROR-γt+(D) and CD8+/IFN-γ+(E). Tumor mass in 8 mice per group was assessed with calipers until 10% of body mass or 14 days of growth (F).

FIG. 25 (A) is a Western blot showing PFKFB3 expression is induced in murine monocytic MDSCs. CD11b+GR-1dim Ly-6G⁻ monocytic MDSCs (M-MDSC) were isolated from spleens of C57BL/6 mice bearing a s.c. B16-F10 melanoma tumor. PFKFB3 protein expression was analyzed in M-MDSC and in monocytes isolated from spleens of naïve mice. (B) is a Western blot showing PFKFB3 expression is induced in established melanoma cell line-educated monocytic MDSCs. MDSCs were isolated from A375:monocyte co-cultures (A375-MDSC). PFKFB3 protein expression was analyzed in A375-MDSCs and in monocytes cultured in the absence of tumor cells.

FIG. 26 (A) shows histograms showing changes in T cell proliferation when T cells are exposed to two different ratios (1:1 and 1:2) of untreated or PFK-158-treated MDSC (during 64 hours of tumor-monocyte co-culture) or to normal fresh monocyte cells or to cultured monocyte cells. (B) shows bar graphs showing changes in T cell proliferation when T cells are exposed as described in part (A). (C) shows bar graphs showing IFN-γ expression in T cells that are exposed to the same conditions.

FIG. 27 (A) shows histograms showing changes in T cell proliferation when T cells are exposed to untreated or PFK-158-treated MDSC (for 24 hours at two doses of the drug: 2.5 μM and 5.0 μM). (B) is a bar graph showing changes in T cell proliferation when T cells are exposed to the same conditions. (C) is a bar graph showing IFN-γ expression in T cells that are exposed to the same conditions.

FIG. 28 shows antigen-specific T cell Suppressive function with Murine MDSCs: Murine M-MDSCs and not G-MDSCs derived from spleens of B16-F10 tumor-bearing mice are suppressive and the antigen-specific suppressive function of M-MDSCs is reversed following ex vivo treatment with PFK-158. (A) shows the M-MDSC to OT-II splenocyte ratio; (B) shows the G-MDSC to OT-II splenocyte ratio.

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “subject” refers to any mammalian subject, including mice, rats, rabbits, pigs, non-human primates, and humans.

The term “cancer” as used herein refers to diseases caused by uncontrolled cell division and the ability of cells to metastasize, or to establish new growth in additional sites. The terms “malignant,” “malignancy,” “neoplasm,” “tumor,” and variations thereof refer to cancerous cells or groups of cancerous cells.

Specific types of cancer include, but are not limited to, melanoma, glioblastoma multiforme, skin cancers, connective tissue cancers, adipose cancers, breast cancers, lung cancers, stomach cancers, pancreatic cancers, ovarian and reproductive organ cancers, cervical cancers, uterine cancers, anogenital cancers, kidney cancers, bladder cancers, liver cancers, colorectal or colon cancers and digestive (GI) tract cancers, prostate cancers and reproductive organ cancers, central nervous system (CNS) cancers, retinal cancer, blood, and lymphoid cancers, and head and neck cancers.

The term “therapeutically effective amount” refers to an amount of a composition high enough to significantly positively modify the symptoms and/or condition to be treated, such as by inhibiting or reducing the proliferation of, or inducing cell death of dysplastic, hyperproliferative, or malignant cells or by abrograting an autoimmune disorder, but low enough to avoid serious side effects (at a reasonable risk/benefit ratio), within the scope of sound medical judgment. The therapeutically effective amount of agents for use in the compositions and methods of the invention herein will vary with the particular condition being treated, the age and physical condition of the patient to be treated, the severity of the condition, the duration of the treatment, the nature of concurrent therapy, the particular agents(s) being employed, the particular pharmaceutically-acceptable carriers utilized, and like factors within the knowledge and expertise of the attending physician.

The term “carrier,” as used herein, includes pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol, and PLURONICS™.

The terms “treat,” “treatment,” and “treating,” as used herein, refer to a method of alleviating or abrogating a disease, disorder, and/or symptoms thereof.

### Methods of Use

The present disclosure demonstrates that Th17 cells and γδT17 cells require high PFKFB3 activity and that PFK-158, a PFKFB3 inhibitor, decreases Th17 cells and γδT17 cells in B16-F10 melanomas and cancer patients and increases the pre-clinical activity of anti-CTLA-4 treatment.

The present disclosure indicates that PFKFB3 expression is required for Th17 and γδT17 cell IL-17 production and may prove to be an effective target to induce anti-tumor immunity via suppression of myeloid derived suppressive cells (MDSCs). Importantly, this runs counter to the current previously held belief that activated T cells require PFKFB3 and that PFKFB3 inhibitors will be immunosuppressive in nature. Further, no published research articles have demonstrated that PFKFB3 inhibition promotes the expansion of effector CD8⁺ T cells or increases the activity of ICIs as disclosed herein. Accordingly, while not desiring to be bound by theory, the data presented herein indicate that PFKFB3 is selectively required for Th17/γδT17 cells and that PFKFB3 inhibitors may be useful to activate anti-tumor immunity, particularly in combination with immune checkpoint inhibitors.

Immune checkpoint inhibitors include agents that inhibit CTLA-4, PD-1, PD-L1, and the like. Suitable anti-CTLA-4 therapy agents for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, ipilimumab, tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res, 58:5301-5304 (1998), U.S. Pat. Nos. 5,977,318, 6,682,736, 7,109,003, and 7,132,281.

Suitable anti-PD-1 and anti-PD-L1 therapy agents for use in the methods of the invention, include, without limitation, anti-PD-1 and anti-PD-L1 antibodies, human anti-PD-1 and anti-PD-L1 and anti-PD-L1 antibodies, mouse anti-PD-1 and anti-PD-L1 antibodies, mammalian anti-PD-1 and anti-PD-L1 antibodies, humanized anti-PD-1 and anti-PD-L1 antibodies, monoclonal anti-PD-1 and anti-PD-L1 antibodies, polyclonal anti-PD-1 and anti-PD-L1 antibodies, chimeric anti-PD-1 and anti-PD-L1 antibodies. In specific embodiments, anti-PD-1 therapy agents include nivolumab, pembrolizumab, pidilizumab, MEDI0680, and combinations thereof. In other specific embodiments, anti-PD-L1 therapy agents include atezolizumab, BMS-936559, MEDI4736, MSB0010718C, and combinations thereof.

Suitable anti-PD-1 and anti-PD-L1 antibodies are described in Topalian, et al., Immune Checkpoint Blockade: A Common Denominator Approach to Cancer Therapy, Cancer Cell 27: 450-61 (Apr. 13, 2015), incorporated herein by reference in its entirety.

Combination treatments involving PFK-158 and an immune checkpoint inhibitor can be achieved by administering PFK-158 and the immune checkpoint inhibitor at the same time. Such combination treatments can be achieved by administering a single composition or pharmacological formulation that includes both agents, or by administering two distinct compositions or formulations, at the same time, wherein one composition includes PFK-158 and the other includes the immune checkpoint inhibitor.

Alternatively, treatment with PFK-158 can precede or follow treatment with the immune checkpoint inhibitor by intervals ranging from minutes to weeks. In embodiments where the immune checkpoint inhibitor and PFK-158 are administered separately, one would generally ensure that a significant period of time did not expire between the time of each delivery, such that the immune checkpoint inhibitor and PFK-158 treatment would still be able to exert an advantageously combined effect. In such instances, it is provided that one would contact the cell with both modalities within about 12-24 hours of each other and, optionally, within about 6-12 hours of each other. In some situations, it can be desirable to extend the time period for treatment significantly, however, where several days (2, 3, 4, 5, 6 or 7) to several weeks (1, 2, 3, 4, 5, 6, 7 or 8) lapse between the respective administrations. Also, under some circumstances, more than one administration of either PFK-158 or of the immune checkpoint inhibitor will be desired.

In one embodiment, a method of treating cancer is provided, the method comprising administering to a subject in need thereof a synergistic, therapeutically effective amount of (1) (E)-1-(pyridyn-4-yl)-3-(7-(trifluoromethyl)quinolin-2-yl)-prop-2-en-1-one (PFK-158); and (2) an immune checkpoint inhibitor. In some embodiments, the immune checkpoint inhibitor is an anti-CTLA-4 antibody selected from the group consisting of ipilimumab, tremelimumab, and combinations thereof. In a specific embodiment, the immune checkpoint inhibitor is ipilimumab.

In another embodiment, the immune checkpoint inhibitor is an anti-PD-1 antibody selected from the group consisting of nivolumab, pembrolizumab, pidilizumab, MEDI0680, and combinations thereof. In another embodiment, the immune checkpoint inhibitor is an anti-PD-L1 antibody selected from the group consisting of atezolizumab, BMS-936559, MEDI4736, MSB0010718C, and combinations thereof.

In still another embodiment, PFK-158 is administered with one or more immune checkpoint inhibitors selected from the group consisting of ipilimumab, tremelimumab, nivolumab, pembrolizumab, pidilizumab, MEDI0680, atezolizumab, BMS-936559, MEDI4736, MSB0010718C, and combinations thereof.

In another embodiment, a method of stimulating anti-tumor immunity in a subject in need thereof is provided, comprising administering to the subject a therapeutically effective amount of (E)-1-(pyridyn-4-yl)-3-(7-(trifluoromethyl)quinolin-2-yl)-prop-2-en-1-one (PFK-158). In some embodiments, the method further comprises administering an effective amount of an immune checkpoint inhibitor. In some embodiments, the immune checkpoint inhibitor is an anti-CTLA-4 antibody. In other embodiments, the immune checkpoint inhibitor is one ore more selected from the group consisting of ipilimumab, tremelimumab, nivolumab, pembrolizumab, pidilizumab, MEDI0680, atezolizumab, BMS-936559, MEDI4736, MSB0010718C, and combinations thereof.

Also provided is a method of synergistically increasing the activity of an immune checkpoint inhibitor comprising administering to a subject in need thereof synergistic, therapeutically effective amount of (1) (E)-1-(pyridyn-4-yl)-3-(7-(trifluoromethyl)quinolin-2-yl)-prop-2-en-1-one (PFK-158) and (2) the immune checkpoint inhibitor. In certain embodiments, the immune checkpoint inhibitor is an anti-CTLA-4 therapy selected from the group consisting of ipilimumab, tremelimumab, and combinations thereof. In a specific embodiment, the immune checkpoint inhibitor is ipilimumab. In another embodiment, the immune checkpoint inhibitor is an anti-PD-1 therapy selected from the group consisting of nivolumab, pembrolizumab, pidilizumab, MEDI0680, and combinations thereof. In still another embodiment, the immune checkpoint inhibitor is an anti-PD-L1 therapy selected from the group consisting of atezolizumab, BMS-936559, MEDI4736, MSB0010718C, and combinations thereof. In still another embodiment, PFK-158 is administered with one or more immune checkpoint inhibitors selected from the group consisting of ipilimumab, tremelimumab, nivolumab, pembrolizumab, pidilizumab, MEDI0680, atezolizumab, BMS-936559, MEDI4736, MSB0010718C, and combinations thereof.

Also provided herein is a method of immunotherapy comprising administering to a subject in need thereof a therapeutically effective amount of (E)-1-(pyridyn-4-yl)-3-(7-(trifluoromethyl)quinolin-2-yl)-prop-2-en-1-one (PFK-158). As with previous methods described herein, the PFK-158 is optionally administered together with an immune checkpoint inhibitor, such as anti-CTLA-4, anti-PD-1, anti-PD-L1, and combinations thereof. In certain embodiments, PFK-158 is administered with one or more immune checkpoint inhibitors selected from the group consisting of ipilimumab, tremelimumab, nivolumab, pembrolizumab, pidilizumab, MEDI0680, atezolizumab, BMS-936559, MEDI4736, MSB0010718C, and combinations thereof.

Strong evidence implicates the Th17 lineage in several autoimmune disorders. Hence, a drug that depletes Th17 cells, such as PFK-158, may have utility for the treatment of a variety of autoimmune diseases, including lupus, rheumatoid arthritis, multiple scleroisis, ulcerative colitis, inflammatory bowel disease, asthma, Crohn's disease, psoriasis, and diabetes mellitus type 1. See, for example, Bedoya, et al., Th17 Cells in Immunity and Autoimmunity, Clinical and Developmental Immunology 2013: Article ID 986789 (2013), incorporated herein by reference in its entirety.

### Pharmaceutical Compositions

The PFKFB3 inhibitors, such as PFK-158, and immune checkpoint inhibitors described herein are all referred to herein as “active compounds.” Pharmaceutical formulations comprising the aforementioned active compounds also are provided herein. These pharmaceutical formulations comprise active compounds as described herein, in a pharmaceutically acceptable carrier. Pharmaceutical formulations can be prepared for oral or intravenous administration as discussed in greater detail below. Also, the presently disclosed subject matter provides such active compounds that have been lyophilized and that can be reconstituted to form pharmaceutically acceptable formulations (including formulations pharmaceutically acceptable in humans) for administration.

The therapeutically effective dosage of any specific active compound, the use of which is within the scope of embodiments described herein, will vary somewhat from compound to compound, and subject to subject, and will depend upon the condition of the subject and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once per day for a period of two to three weeks or until the condition is essentially controlled. Lower doses given less frequently can be used prophylactically to prevent or reduce the incidence of recurrence of the infection.

It is appreciated that the doses will vary, depending on the particular active agent and the condition to be treated. For example, if the subject is administered ipilimumab intravenously, a dose can vary from about 3 mg/kg (for stage IV melanoma) to about 10 mg/kg (for stage III melanoma). With respect to nivolumab, the intravenous dose can vary from 1-3 mg/kg for multiple indications. With respect to pembrolizumab, the intravenous dose can vary from 1-3 mg/kg, more specifically about 2 mg/kg for multiple indications. PFK-158 is administered intravenously at a dose of from about 10 to about 1000 mg/m², more specifically from about 10 to about 700 mg/m², and more specifically about 24 to about 650 mg/m².

In accordance with the presently disclosed methods, pharmaceutically active compounds as described herein can be administered orally as a solid or as a liquid, or can be administered intramuscularly or intravenously as a solution, suspension, or emulsion. Alternatively, the compounds or salts also can be administered intravenously or intramuscularly as a liposomal suspension.

Pharmaceutical formulations suitable for intravenous or intramuscular injection are further embodiments provided herein. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is preferably done by an aseptic method. Sterilized closures can then be placed on the vials and, if desired, the vial contents can be lyophilized.

In addition to PFK-158 and an immune checkpoint inhibitor, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. The antimicrobial preservative is typically employed when the formulation is placed in a vial designed for multi-dose use. The pharmaceutical formulations described herein can be lyophilized using techniques well known in the art.

In yet another embodiment of the subject matter described herein, there is provided an injectable, stable, sterile formulation comprising PFK-158 and an immune checkpoint inhibitor in unit dosage form in a sealed container. The active compounds are provided in the form of a lyophilizate, which is capable of being reconstituted with a suitable pharmaceutically acceptable carrier to form a liquid formulation suitable for injection thereof into a subject. When the active compounds are substantially water-insoluble, a sufficient amount of emulsifying agent, which is physiologically acceptable, can be employed in sufficient quantity to emulsify the compound or salt in an aqueous carrier.

In one embodiment, a pharmaceutical composition is provided, comprising: (a) a therapeutic amount of (E)-1-(pyridyn-4-yl)-3-(7-(trifluoromethyl)quinolin-2-yl)-prop-2-en-1-one (PFK-158); (b) a therapeutic amount of at least one immune checkpoint inhibitor; and (c) at least one pharmaceutically-acceptable carrier. In certain embodiments, the immune checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C, and combinations thereof. In other embodiments, the immune checkpoint inhibitor is a therapeutic agent selected from the group consisting of anti-CTLA-4, anti-PD-1, anti-PD-L, and combinations thereof.

### EXAMPLES

The presently disclosed subject matter will be described more fully hereinafter with reference to the accompanying Examples, in which representative embodiments are shown. The presently disclosed subject matter can, however, be embodied in different forms and should not be construed as limited to the embodiments set forth herein.

### Example 1

### Small Molecule Inhibition of PFKFB3 with PFK-158 Reduces the Frequencies of Tumor-Infiltrating Th17 and γδ17 Cells, MDSCs, and Regulatory T Cells (Treg) and Increases the Frequencies of Intratumoral CD4+ and CD8+/IFN-γ+ T Cells and B Cells

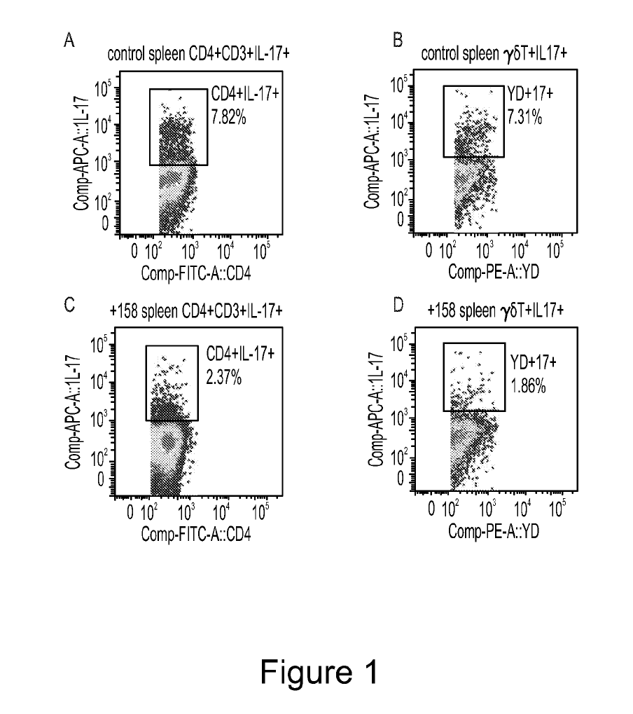

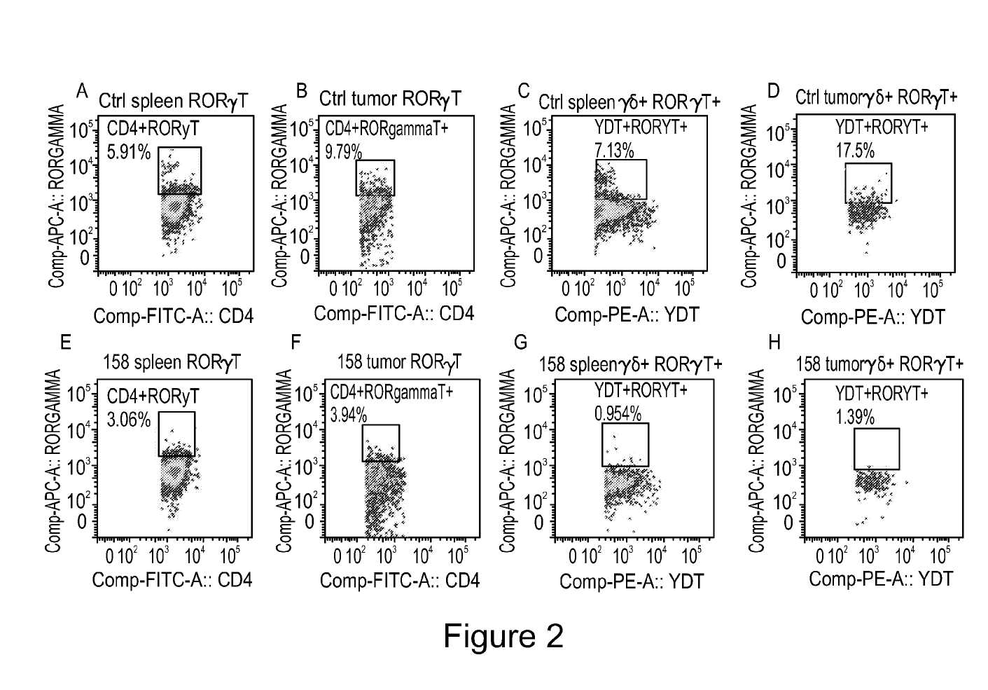

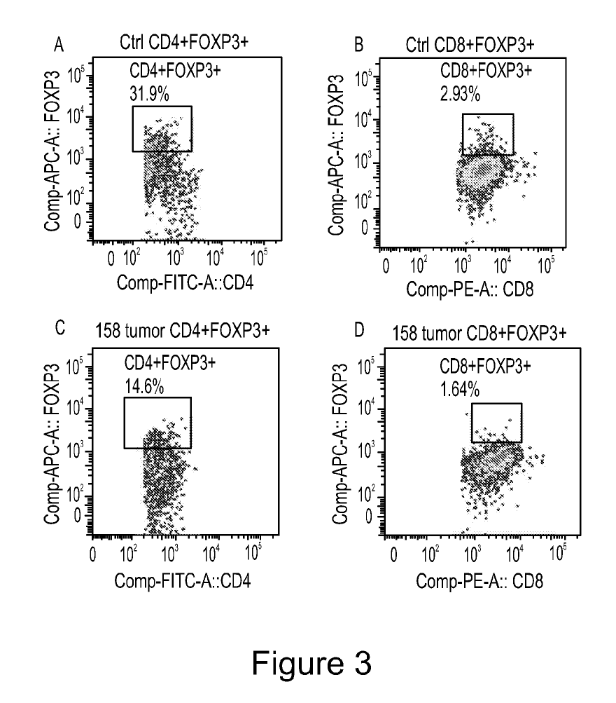

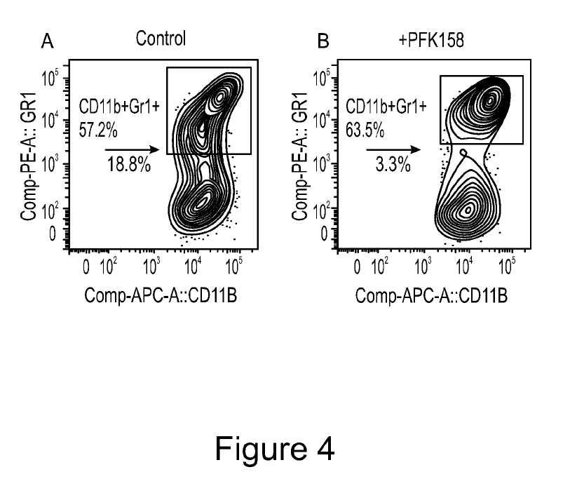

The effect of PFK-158 (60 mg/kg IP×3 days) on splenic and tumor infiltrating immune cells in mice bearing B16-F10 melanoma tumors was examined. A panel of cell types was studied that encompassed both immunosuppressive cells (Th17 cells, γδT17 cells, regulatory T (Treg) cells, and myeloid derived suppressive cells (MDCSs) and immune activating cells including CD4+ and CD8+ T cells. Surprisingly, a marked decrease was observed in splenic Th17 and γδT17 cells using intracellular cytokine staining for IL-17 (FIG. 1) as well as splenic and intratumoral Th17 cells and γδT17 cells using the retinoid-related orphan receptor-γδ as a marker for the Th17 cells and γδT17 cells (FIG. 2). A marked decrease was observed in the immunosuppressive intratumoral Treg CD4+ and CD8+ cells using FoxP3 as a marker (FIG. 3) as well as reduced intratumoral functional MDSCs (intermediate GR1) after PFK-158 administration (FIG. 4).

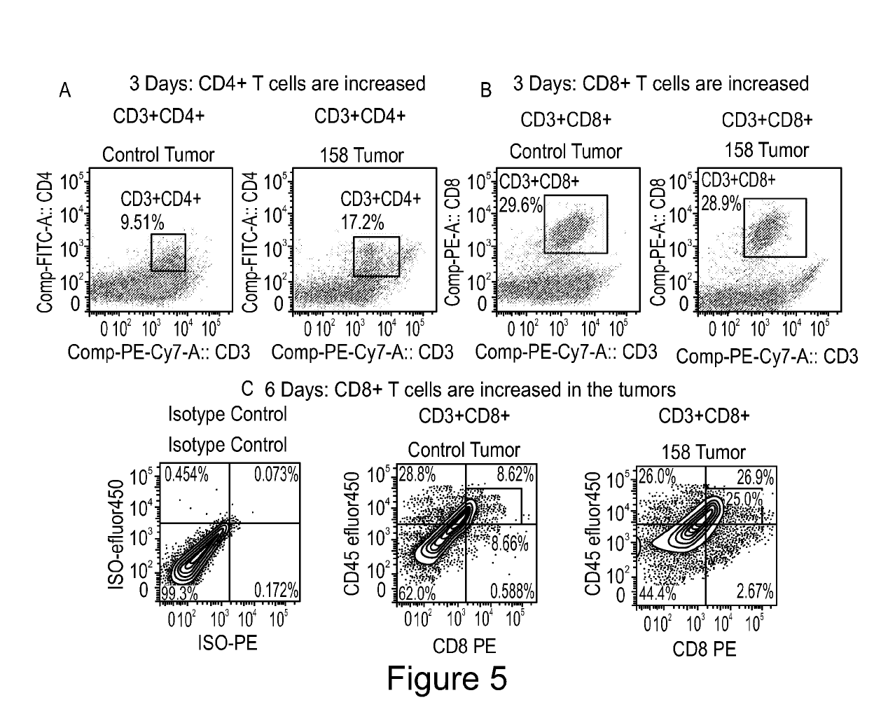

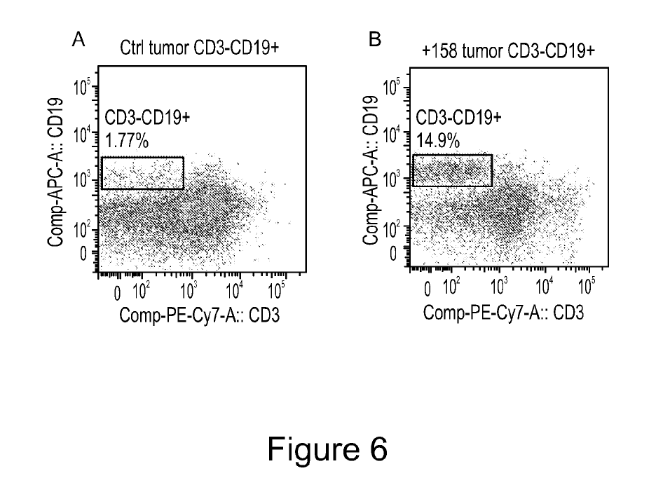

Next, whether this reduction in immunosuppressive cells caused any change in the immune effector cells was examined. Results showed that both CD4+ T cells and CD8+ were unexpectedly increased in the tumors (FIG. 5). Furthermore, a marked increase was observed in CD19+ B cells in the tumors, which can produce anti-tumor antibodies, induce T cell proliferation with cytokines, mediate direct tumor killing via granzymes and present tumor antigens to T cells (FIG. 6).

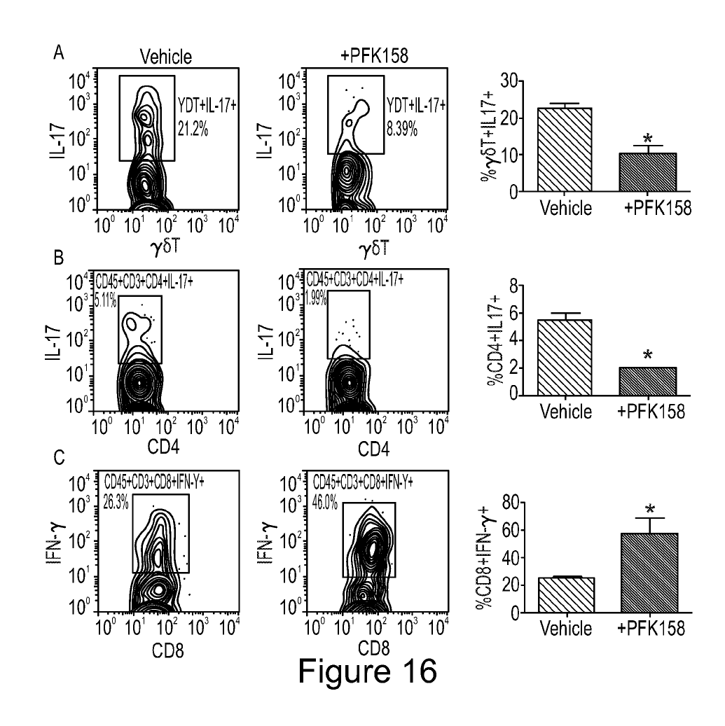

Given the surprisingly potent effect of PFK-158 on Th17 differentiation and γδT17 polarization in vitro, the effect of PFK-158 administration on the reduction of the frequencies of these cells in B16-F10 melanomas was studied. 0.06 mg/gm PFK-158 was administered i.p.×3 days to mice bearing 100 mg tumors, mice were sacrificed and the tumor-infiltrating Th17 and γδT17 cells were quantified using intracellular cytokine staining for IL-17. A reduction in the percentages of tumor-infiltrating Th17 and γδT17 cells was observed after PFK-158 administration (FIG. 16 & Table 1).

Th17 cells and/or γδT17 cells reduce the effector CD8⁺ T function in tumors by promoting MDSC accumulation. We thus hypothesized that a reduction in tumor-infiltrating Th17 and γδT17 cells caused by PFK-158 administration would increase intra-tumoral CD8⁺/IFNγ⁺ T cells. We observed a marked increase in CD8⁺/IFNγ⁺ T cells in tumors after 3 days of PFK-158 administration (FIG. 16 and Table 1, below).

B16-F10 tumor-bearing (100 mg) C57BL/6 mice were administered 0.06 mg/gm PFK-158 i.p.×3 days, euthanized and the tumor-infiltrating immune cells were analyzed by flow cytometry. For intracellular cytokine staining, cells were activated with PMA/ionomycin for 6 hours and then stained for surface markers/intracellular IL-17. Percentages of Th17 and γδT17 cells were decreased (FIG. 16A, 16B) whereas percentages of CD8+IFN-γ⁺ T cell were increased in the tumors after PFK-158 administration (FIG. 16C). n=3; *p value <0.01

8 B16-F10 tumor-bearing (100 mg) C57BL/6 mice were administered 0.06 mg/gm PFK-158 i.p.×3d, euthanized and the tumor-infiltrating cells were analyzed by FACS. The experiment was repeated 3× and the ave±SD is presented in Table 1 (p value <0.01).

TABLE 1
Effects of PFK-158 on immunosuppressive
and effector lymphocytes

| Cell Type | +PFK-158 (% change) |
|---|---|
| Th17 cells | ↓62 ± 11 |
| γδT17 cells | ↓61 ± 7 |
| M-MDSCs | ↓83 ± 9 |
| CD4+/Foxp3+ | ↓54 ± 3 |
| CD4+ | ↑81 ± 11 |
| CD8+/IFNγ+ | ↑75 ± 14 |
| CD19+ | ↑58 ± 9 |

MDSCs suppress CD8⁺ T cell function in part via the increased activity of arginase which reduces local L-arginine required not only for CD8⁺ T cells but also for CD4⁺ T cells and B cells. A reduction in MDSCs was observed that correlated with an increase in not only tumor-infiltrating CD8+/IFN-γ+ T cells but also CD4⁺ T and B cells (Table 1). Suppression of HIF-1a promotes the differentiation of T_{reg} in vitro and thus an increase was expected in these cells; rather, it was observed that they were reduced in the B16-F10 tumors (Table 1). Given that MDSCs promote T_{reg} cells via IL-10, TGFβ and arginase, and that PFK-158 reduced tumor-infiltrating MDSCs, it is believed that the observed reduction in T_{reg} cells is caused by a depletion of MDSCs in the tumor. In summary, these data indicate that the net effect of small molecule inhibition of PFKFB3 on the tumor immune micro-environment is to reduce the percentages of Th17 cells, γδT17 cells, MDSCs and T_{reg} cells and to increase the percentages of tumor-infiltrating CD4⁺ and CD8+/IFNγ+ T cells, and B cells. Furthermore, these data indicate that PFKFB3 inhibition may have potential to induce immune-mediated tumor regressions.

### Example 2

### PFK-158 Increases the Anti-Tumor Activity of Anti-CTLA-4 in B16-F10 Melanoma-Bearing Mice

In view of the fact that PFK-158 monotherapy decreases tumor-infiltrating immunosuppressive cells and increases tumor-infiltrating CD8+/IFNγ+ T cells, the effect of PFK-158 on the anti-tumor activity of the immune checkpoint inhibitor anti-CTLA-4 antibody was studied. PFK-158 (0.06 mg/gm i.p.×3 days and then every other day) was combined with an anti-CTLA-4 antibody 9D9 (0.1 mg i.p. every third day×3) and the growth of established B16-F10 melanoma tumors (100 mg) in C57BL/6 mice was examined. 9D9 is an anti-CTLA-4 antibody that has shown efficacy in B16 melanoma models. See Curran, et al., PD-1 and CTLA-4 Combination Blockade Expands Infiltrating T Cells and Reduces Regulatory T and Myeloid Cells within B16 Melanoma Tumors, PNAS 107(9): 4275-80 (Mar. 2, 2010), incorporated herein by reference in its entirety.

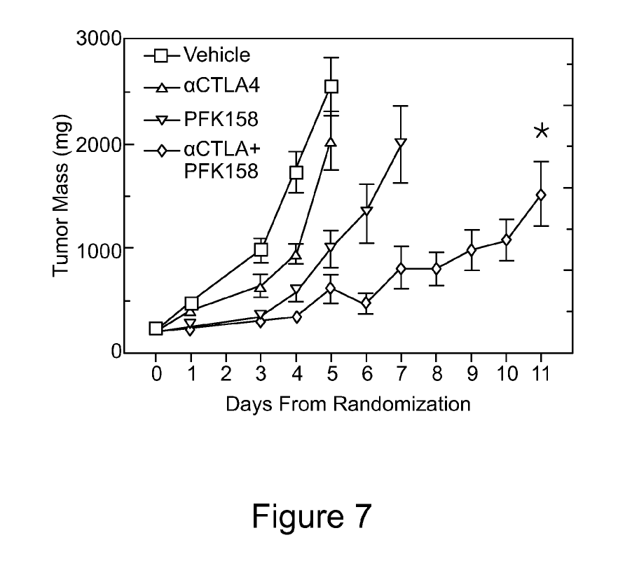

A synergistic increase in anti-tumor activity that surpassed the additive effects of each therapy alone was observed by day 7 of treatment (FIG. 7). These data indicated that selective PFKFB3 inhibition may prove to be a useful strategy to not only increase tumor-infiltrating CD8+/IFNγ+ T cells, but also to potentiate the effects of immune checkpoint inhibitors in order to further improve clinical outcomes of cancer patients.

### Example 3

### Effects of PFK-158 on Th17 Cells, γδT-17 Cells, Treg Cells, Activated CD4+T and CD8+ T Cells

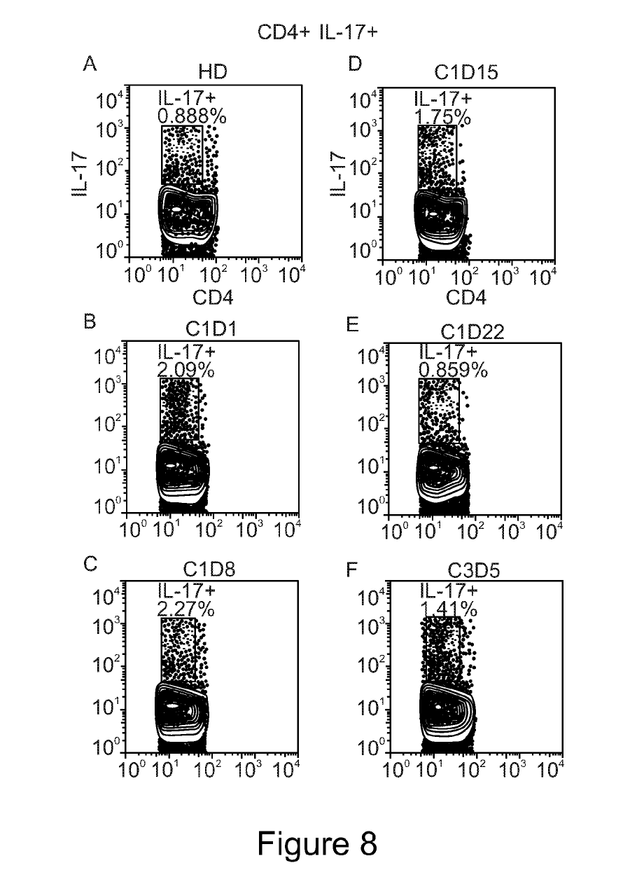

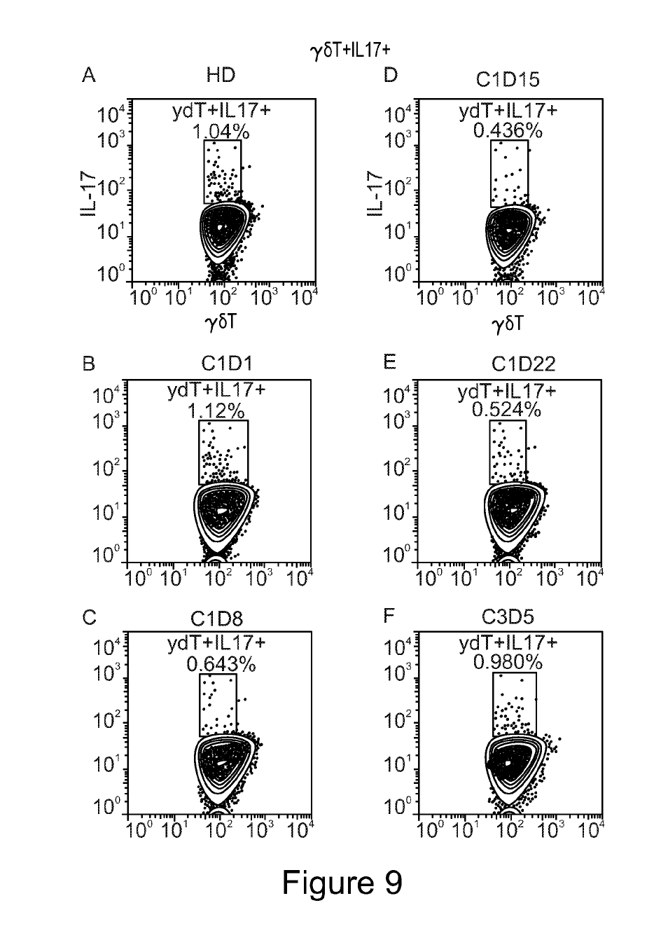

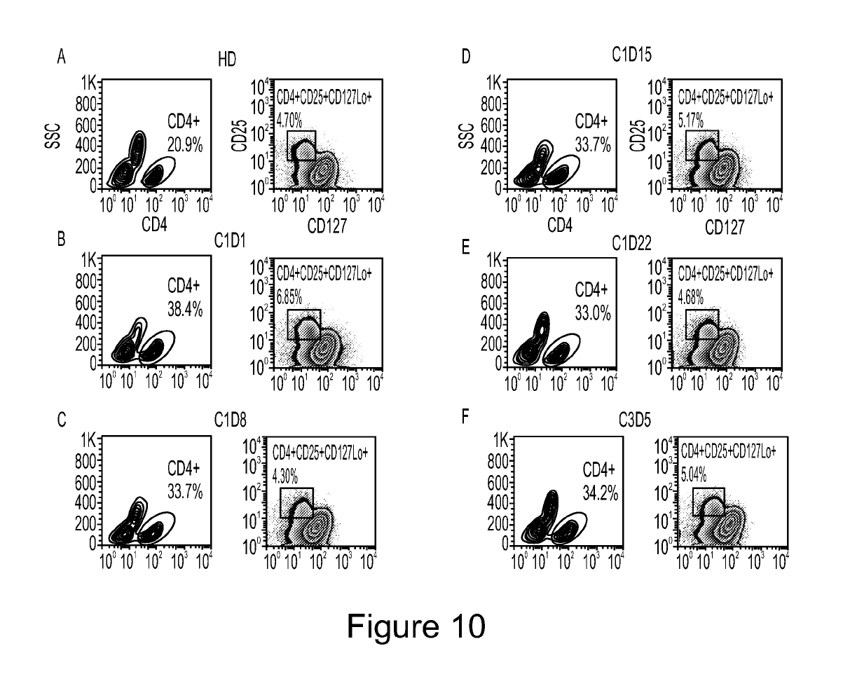

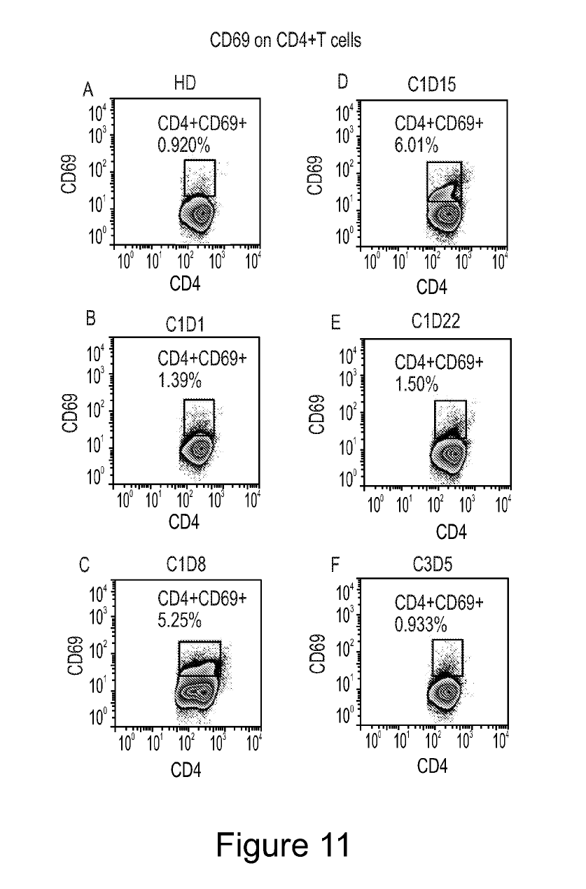

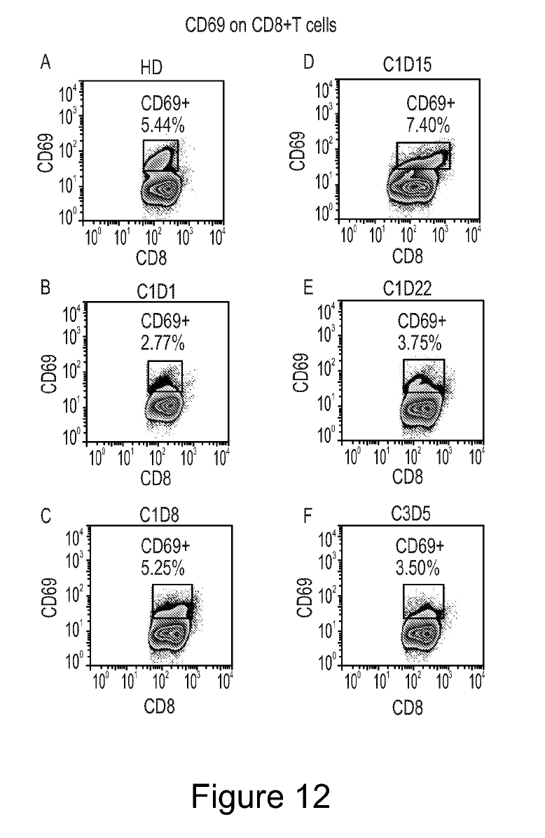

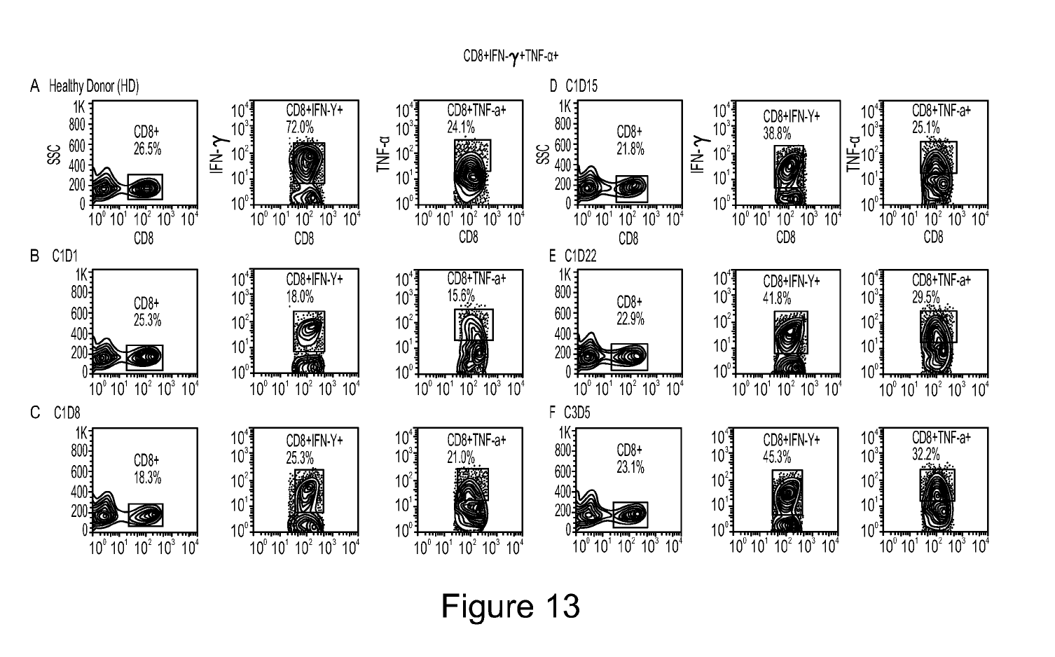

An unexpected stability of tumors in multiple patients receiving PFK-158 at doses that were considered to be sub-therapeutic has been observed. Based on data demonstrating that PFK-158 appears to have a paradoxical stimulatory effect on anti-tumor immunity, the peripheral blood of a human subject receiving PFK-158 was examined. This breast adenocarcinoma patient received 96 mg/M² of PFK-158 every other day for three weeks followed by a one week rest—this cycle was repeated for a total of four cycles. Peripheral blood mononuclear cells were collected on days 1, 8, 15, 22 and 62 and flow cytometry was conducted for a multitude of immunosuppressive cells and activated T cells. Initially, the immunosuppressive cells in the peripheral blood at baseline (day 1; C1D1) and after PFK-158 administration (days 8 (C1D8), 15 (C1D15), 22 (C1D22) and 62(C3D5) were examined and reductions were observed in Th17 cells (FIG. 8; HD=healthy donor control), γ6T-17 cells (FIG. 9) and Treg cells (FIG. 10). The concentration of activated CD4+ and CD8+ T cells was examined using CD69, which is the TNFα receptor. PFK-158 surprisingly caused a marked increase activated CD4+ and CD8+ T cells particularly after two cycles of treatment (FIGS. 11 and 12). Further, a marked increase in activatable CD8+ T cells was observed (measured using intracellular staining for interferon-γ and tumor necrosis factor-α after in vitro activation with PMA/ionomycin) (FIG. 13). These cells are the main CD8+ T cells that can infiltrate tumors and differentiate into cytolytic T cells which kill tumor cells.

### Example 4

### PFKFB3 Inhibition Via PFK-158 Increases Anti-Tumor Specific Immunity

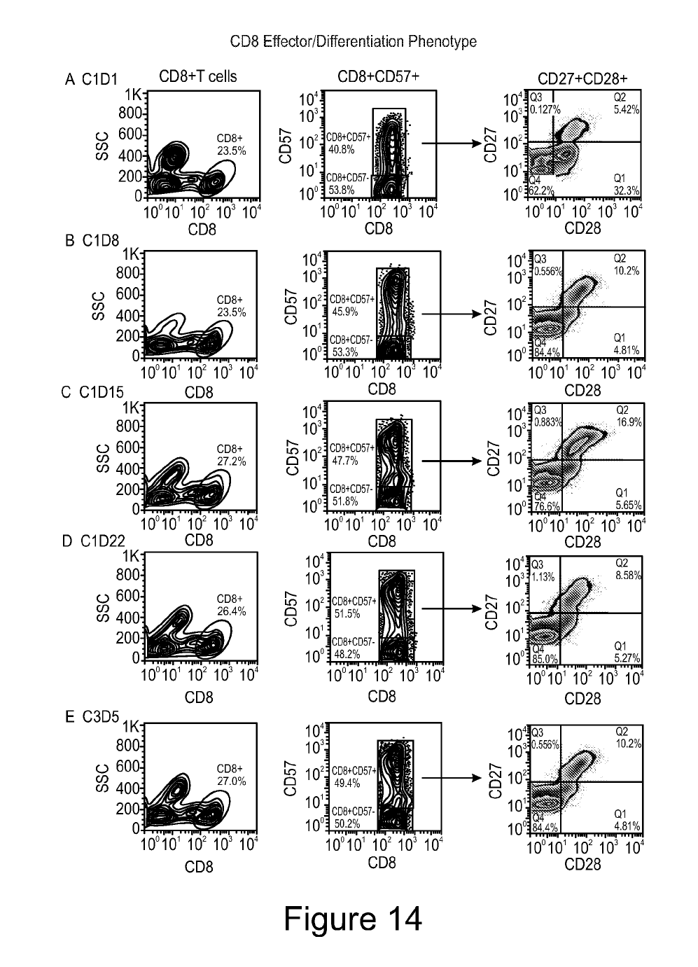

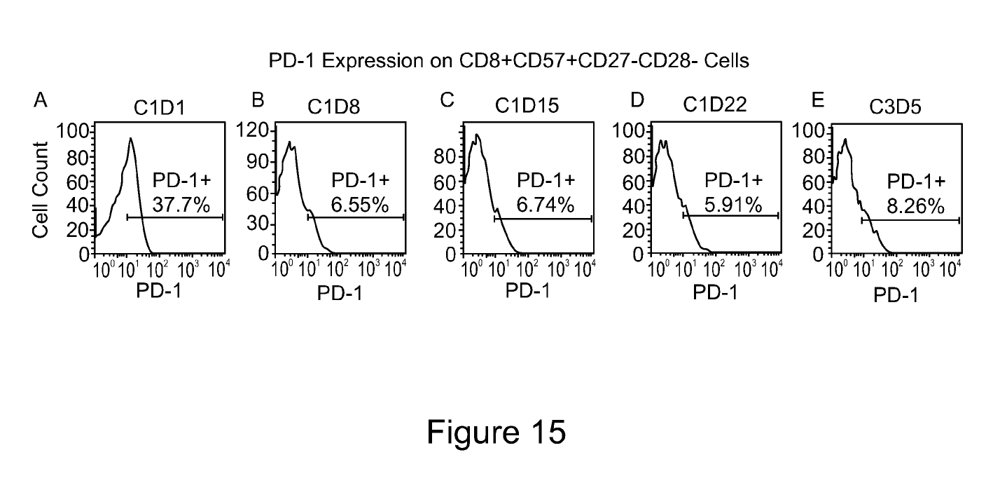

In cancer patients, a distinct population of CD8+ arises that form the main tumor infiltrating cytolytic T cells—these cells are CD8+/CD57+/CD27-/CD28- and express the immune checkpoint protein PD-1 (which attenuates their activity) (Gros, et al., PD-1 identifies the patient-specific CD8(+) tumor-reactive repertoire infiltrating human tumors, J. Clin. Invest. 124:2246-59 (2014)). First, the effect of PFK-158 administration on the circulating concentration of these cancer-reactive CD8+ T cells was examined and results showed a 36% increase, which persisted through the treatment and analysis period (FIG. 14). These data indicate that PFKFB3 inhibition via PFK-158 is increasing anti-tumor-specific immunity. Furthermore, the expression of the immune checkpoint protein PD-1 on these cancer-specific CD8+ T cells was analyzed and results showed that PFK-158 caused a marked decrease in PD-1 expression, which in turn would permit activation and expansion of these essential effector CD8+ T cells (FIG. 15). Importantly, this breast adenocarcinoma patient experienced stabilization of her breast tumors after two months of treatment with PFK-158. Taken together, these human studies complement the mouse studies to demonstrate that PFKFB3 inhibition stimulates anti-tumor immunity via a combination of depletion of immunosuppressive cells and expansion of activated CD4+ and CD8+ T cells.

These results were unexpected and contradictory to the previously held belief that PFKFB3 is required for T cell expansion and thus a target for the development of immunosuppressive agents. The acute effects of PFK-158 in the mice (3 days) and human subject (7 days) on the immune system indicate that PFKFB3 inhibition causes a shift from an immunosuppressive phenotype to an immune activating phenotype and that PFKFB3 inhibitors may be useful as an immunotherapeutic. Accordingly, PFKFB3 inhibitors are useful in methods of (1) stimulating tumor activity and (2) synergistically increase the activity of pre-existing immunotherapies.

### Example 5

### PFKFB3 is Required for the Differentiation and Polarization of Human Th17 and γδT17 Cells

The differentiation of Th17 cells from naïve CD4⁺ T cells was induced and, for γδT17 cells, naïve Vγ9Vδ2 T cells were isolated from healthy donors and polarized into IL-17 producing γ6T cells using established methods.

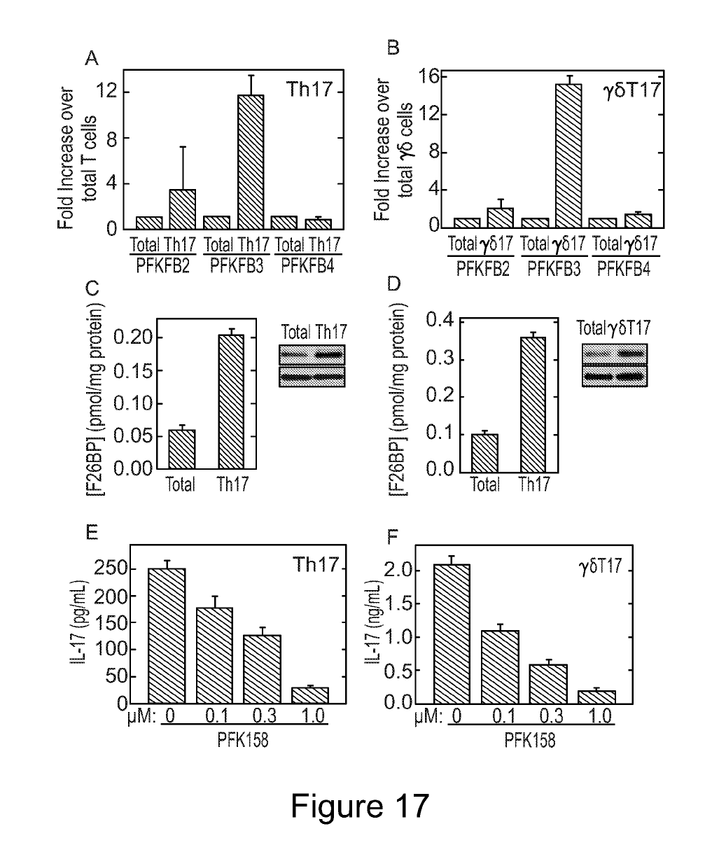

Human naïve CD4⁺ T cells differentiated into Th17 cells or sorted human naïve Vγ9Vδ2 T cells were polarized in vitro and analyzed for PFKFB2-4 mRNA (FIG. 17 A,B), F26BP, and PFKFB3 protein (FIG. 17 C,D). The CD4⁺ T cells (during in vitro Th17 differentiation) and Vγ9Vδ2 T cells (during in vitro polarization) were exposed to vehicle (DMSO) or the indicated concentrations of PFK-158 (every two days of the culture period) and IL-17A production was quantified by ELISA (FIG. 17 E,F).

High IL-17A production was confirmed in both cell types. It was then found that these cells selectively over-express PFKFB3 and maintain a high [F2,6BP], an activator of glucose metabolism needed for growth and survival (FIG. 17A,B and FIG. 17C,E). The effects of the PFKFB3 inhibitor, PFK-158, on Th17 and γδT17 cells was analyzed. It was found that PFK-158 reduced the IL-17A by these cells at a concentration that is ˜10-fold less than required to inhibit neoplastic and endothelial cells growth (FIG. 17E,F). These data suggest that PFKFB3 may be uniquely required for the differentiation/polarization of Th17/γ6T17 cells.

### Example 6

### Small Molecule Inhibition of PFKFB3 with PFK-158 in Human Subjects Reduces the Frequency of Peripheral Blood Th17 Cells and Increases the Frequency of Effector CD8+ T Cells

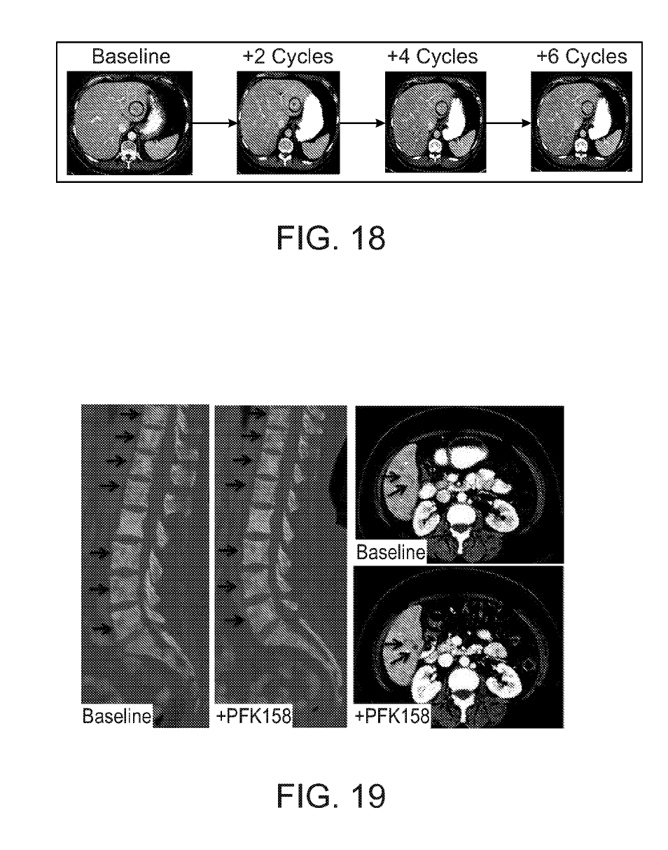

A phase 1 trial of PFK-158 in cancer patients is currently being conducted at Georgetown University, UT Southwestern, Md. Anderson Cancer Center and the U. of Louisville (clinicaltrials.gov# NCT02044861). As of late 2015, the trial has entered cohort 7 without reaching a maximum tolerated dose (currently at 650 mg/M²). Institutional investigators have observed partial tumor regressions in 3 patients with ocular melanoma, ovarian cancer and renal cell carcinoma and de novo stabilization of tumor burden in 2 patients (breast and adenoid cystic carcinoma). A 52 year-old male with ocular melanoma was administered 24 mg/M² in cohort 1 of the phase 1 trial of PFK-158—a dose that is markedly lower than that required to cause the known cytotoxic effects of PFK-158. Despite this low dose, the patient experienced regression of multiple hepatic metastases, in that the majority of the liver lesions are stable to reduced in size and many demonstrate interval decrease in attenuation suggesting necrosis from treatment. Over the course of six months of therapy, several of the patient's hepatic metastases first became necrotic after 2 monthly cycles (hypoattenuation appears dark on CT imaging) and then shrunk after 6 cycles (FIG. 18). These surprising clinical data suggested that PFK-158 may have systemic anti-tumor effects separate from its direct cytotoxic effects on cancer cells.

### Example 7

### PFK-158 Depletes Th17 and γδT17 Cells in Human Subjects and Increases the Activation of CD8⁺ T Cells

The PBMCs were studied from a 51 year old metastatic breast cancer patient who experienced stabilization of her bone metastases and both stabilization and hypoattenuation/necrosis in multiple liver metastases for four months while being administered PFK-158 (FIG. 19). The patient was administered PFK-158 for 4 cycles and her bony metastases were deemed to be stable (FIG. 19, left two panels) and several liver metastases became necrotic (FIG. 19, right panel).

Peripheral blood Th17 cells from a healthy donor and the patient were compared and a greater than two-fold increase was observed in the percentages of peripheral blood Th17 cells in the breast cancer patient. After 8, 15, and 22 days of PFK-158 administration, a cumulative reduction was observed in Th17 cells that essentially normalized the breast cancer patient's peripheral blood (Th17 cell) (data not shown). IL-17 derived from both Th17 and γδT17 cells has been established to promote MDSCs which in turn suppress cytotoxic T cell function and infiltration. The percentages of effector CD8+/IFN-γ+ cells were studied and it was found that 3 weeks of PFK-158 administration doubled the frequencies of these cells in this breast cancer patient (not shown). These data indicate that inhibition of PFKFB3 with PFK-158 in a human subject not only resulted in depletion of Th17 cells but also a marked increase in the circulating percentages of effector CD8⁺ T cells. The TNFR family member, CD137 (4-1BB), is used to identify tumor-reactive T cells from blood and has recently been found to be preferentially expressed on the tumor-reactive subset of tumor-infiltrating lymphocytes. The breast cancer patient's CD8⁺CD137⁺ T cells were analyzed and a marked increase was observed in the first 14 days of PFK-158 administration, but a reduction to near baseline was observed after the 3rd week of PFK-158 administration (not shown). Since these cells are considered to be tumor-reactive CD8⁺ T cells, it was postulated that the observed reduction in the peripheral blood after 3 weeks may be due to homing to metastases.

### Example 8

### PFK-158 Increases CD8+T Effector-Memory Cells that Display a Terminally-Differentiated Effector Phenotype and Reduces their PD-1 Expression

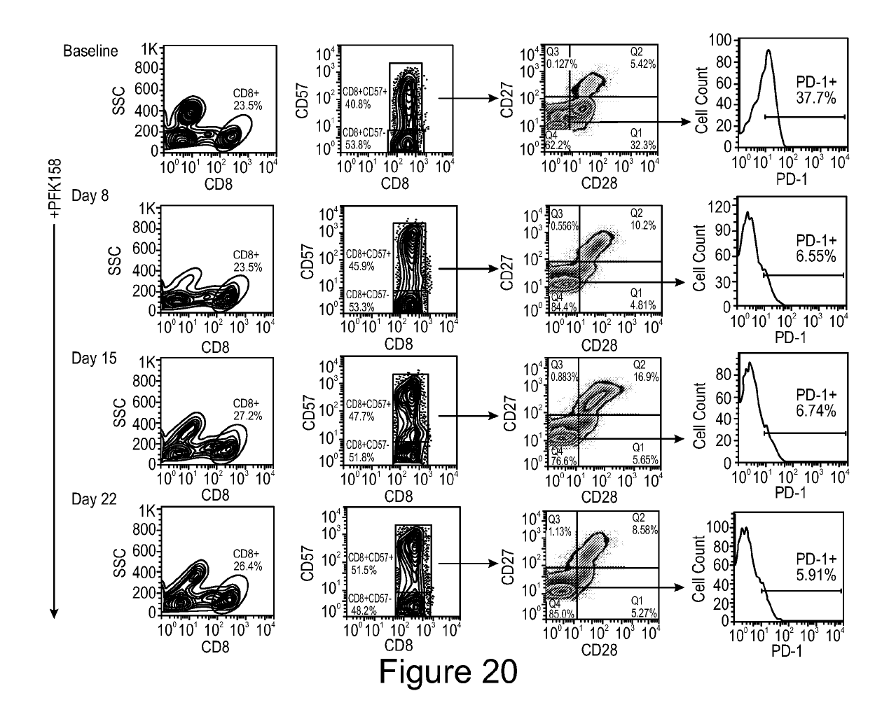

Given the decrease in Th17 cells and increase in effector CD8⁺ T cells observed after PFK-158 administration, the ability of PFK-158 to cause an increase in terminally-differentiated CD8⁺ effector cells was examined. CD8⁺CD57⁺CD27⁻ TCD28⁻ The breast cancer patient described in Example 7 was administered PFK-158 for 1 cycle and PBMCs were collected at baseline, day 8, 15, 22, and then analyzed for CD8⁺CD57⁺CD27⁻ TCD28⁻ T cells and PD-1. T cells are elevated in the tumors and peripheral blood of cancer patients where they are believed to control tumor growth through their antigen-specific cytolytic activity. These tumor-infiltrating CTLs have been found to express PD-1 as a result of chronic antigen stimulation which limits their anti-tumor capacity. Results showed that PFK-158 administration caused a modest 36% increase in CD8⁺CD57⁺CD27⁻ TCD28⁻ T cells after only 1 week and, more importantly, the increase in these cells was concurrent with a near complete loss in the negative regulator PD-1 (FIG. 20).

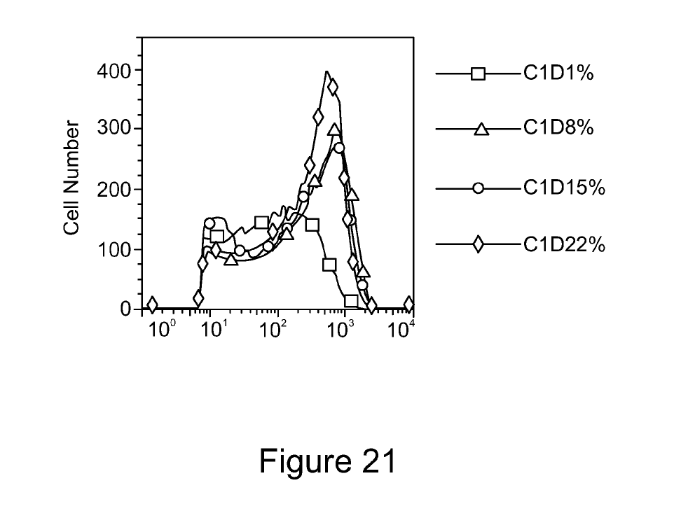

CD57 is a terminally sulfated carbohydrate that is selectively expressed on antigen-specific, and functionally competent memory/effector CD8⁺ T cells. A sub-analysis was conducted of the CD8⁺CD57⁺CD27⁻ TCD28⁻ T cells for relative CD57 expression. PBMCs were collected at baseline, day 8, 15, and 22 and CD8⁺CD57⁺CD27⁻ CD28⁻ T cells were analyzed for CD57 expression. Results showed a positive correlation between CD57 expression and PFK-158 exposure (FIG. 21).

### Example 9

### PFK-158 Simultaneously Decreases Peripheral Blood Immune Suppressor Cell Types and Increases Activated CD4⁺ and CD8⁺ T Cells in a Breast Cancer Patient

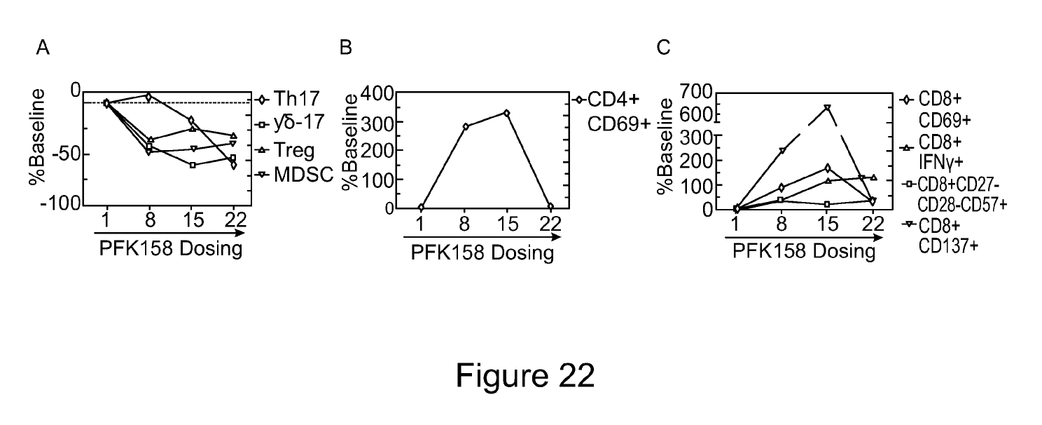

The peripheral blood frequencies of Th17 and γδT17 cells were analyzed simultaneously with monocytic MDSCs (M-MDSC) and an acute drop was observed in all three cell types after PFK-158 administration (FIG. 22A). Interestingly, the reduction in γδT17 cells but not the reduction in Th17 cells coincided with the reduction in MDSCs. Importantly, T_{reg} frequencies also were reduced by PFK-158 as observed in the mouse model (FIG. 22A). Based on the observed reduction in all four immunosuppressive cell types, analysis was broadened and it was found that the CD4⁺CD69⁺ and CD8⁺CD69⁺ T cells were dramatically increased in the first two weeks of PFK-158 administration but returned to baseline after the third week of administration (FIG. 22B, C). As noted above, a similar increase and then decrease in the percentages of CD8⁺CD137⁺ T cells was observed in week 3 but the percentages of effector CD8⁺IFN-γ⁺ T cells continued to increase throughout the administration of PFK-158 (FIG. 22C).

### Example 10

### PFK-158 Consistently Decreases the Frequencies of Th17 Cells in Cancer Patients

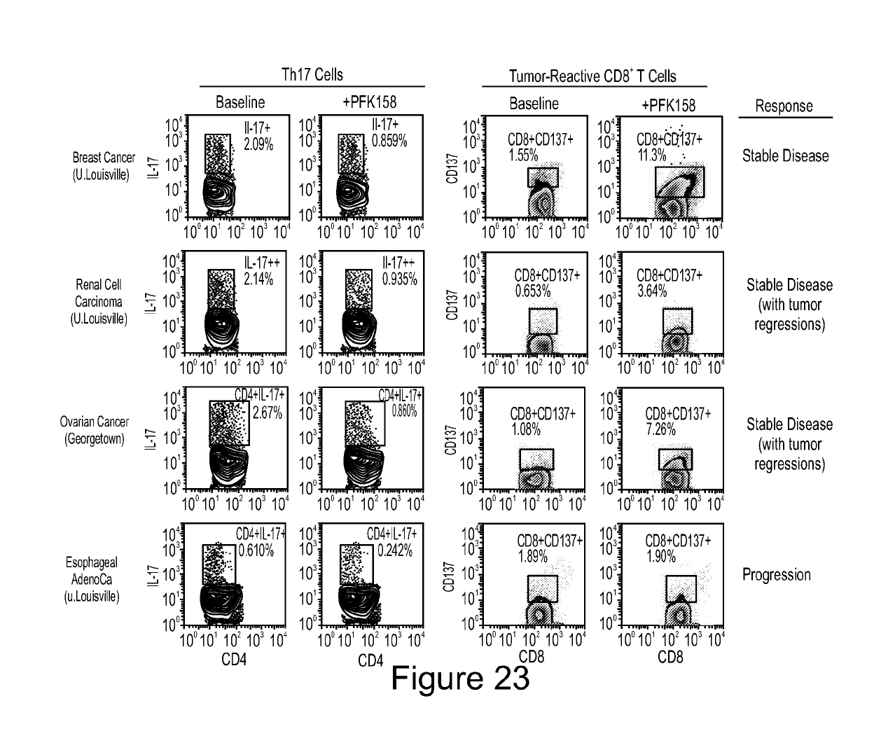

18 cancer patients completed 2 cycles of 24-470 mg/M² PFK-158; 5 of these patients experienced stabilization of their disease (i.e. no growth and no new tumors) and tumor regressions have been observed in multiple patients. For example, a 53 year-old male with renal cell carcinoma who had progressed after all available FDA-approved agents including mTOR and VEGFR inhibitors and IL-2 entered the study (470 mg/M²). He experienced stabilization of disease by immune-related response criteria (26% reduction in maximum diameters) with multiple tumors regressing and has entered his seventh month of treatment. Of these 18 patients, immunophenotyping by flow cytometry on 4 patients' PBMCs has been carried out. These four patients were administered PFK-158 for 2 weeks and PBMCs were collected at baseline, day 15, and then analyzed by flow cytometry for CD3+CD4+il-17+ cells and CD8+CD137+ T cells. After 15 days of PFK158 administration, a reduction in frequency of Th17 cells was observed in all 4 patients, but only ¾ were noted to have an increase in the frequency of the tumor antigen-reactive CD8⁺CD137⁺ T cell subset (FIG. 23). Interestingly, the one patient whose CD8⁺CD137⁺ T cell frequency did not increase also did not have a high baseline frequency of Th17 cells, and progressed after 2 cycles of PFK-158.

Together, these studies indicate that Th17 and γδT17 cells specifically require PFKFB3 for their differentiation, polarization and activity and that selective inhibition of PFKFB3 will deplete these cells and induce tumor-specific immunity.

### Example 11

### Immunological Effects of Genomic Deletion of Pfkfb3 in Host by not in Cancer Cells

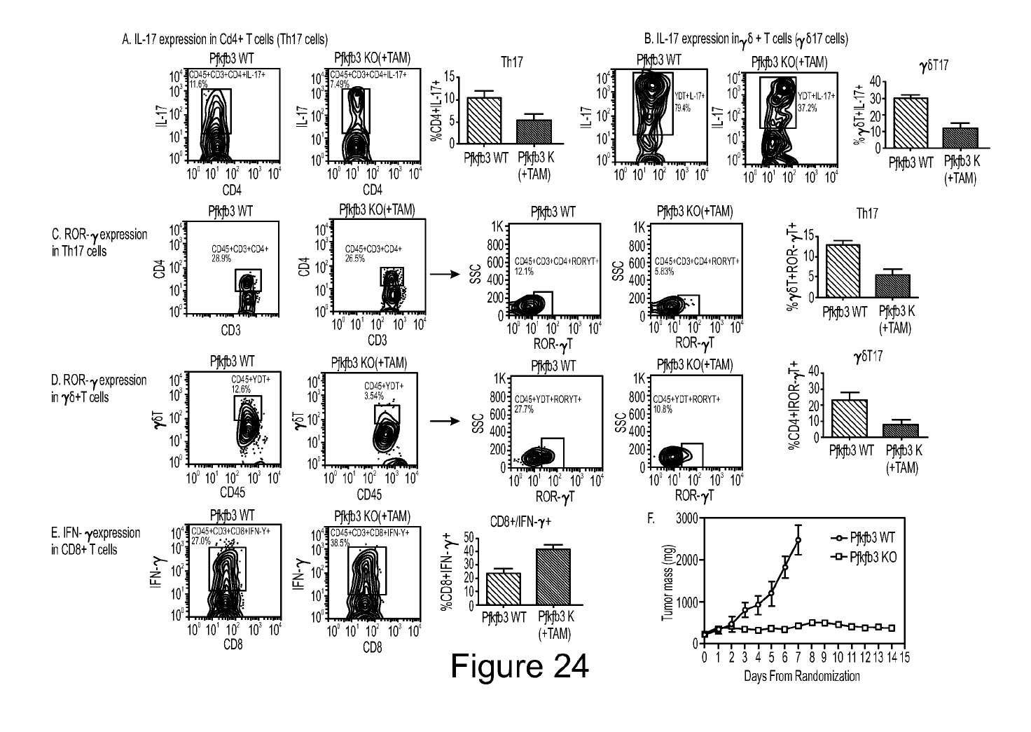

Homozygous Pfkfb3^{fl/fl} mice were crossed with a Cre recombinase strain with a tamoxifen-inducible β-actin promoter to produce Tam-β-actin^{Cre}:Pfkfb3^{fl/fl} mice on a C57BL/6 background. Tamoxifen administration (200 mg/kg, 5 days, i.p.) causes genomic deletion of floxed exons, recombination and decreased PFKFB3 expression and [F26BP] in all organs examined and these mice recently were used to assess the role of PFKFB3 in endothelial cell function. Genomic deletion of Pfkfb3 was induced for 5 days with tamoxifen and then, 2 days later, s.c. injected PFKFB3^{WT} B16 melanoma cells into the Tam-β-actin^{Cre}:Pfkfb3^{fl/fl} and analyzed the tumors from mice that retained wild-type expression of PFKFB3 (Pfkfb3 WT) and those that had undergone genomic deletion of Pfkfb3 (Pfkfb3 KO) (in host cells but not wild-type B16 melanoma cells). In the mice that underwent Pfkfb3 KO (+TAM) in the host but not B16 cells, a marked reduction in tumor growth was observed, reduced Th17 and γδT17 cells and increased CD8⁺ T cells in the tumors—an immune phenotype that was remarkably consistent with that observed after PFK-158 administration (FIG. 24).

Tam-β-Actin^{Cre}:Pfkfb3^{fl/fl} mice at 16 weeks of age were injected with corn oil (Pfkfb3 WT) or tamoxifen (Pfkfb3 KO+TAM, 200 mg/kg×5 days, i.p.) and 2 days later were implanted with 1×10⁵ B16F10 melanoma cells in the flank. 3 mice were euthanized after 4 days for analysis of tumor-infiltrating CD4⁺/IL-17⁺ (FIG. 24A), γδ T+/IL-17⁺ (FIG. 24B), CD4+/ROR-γt⁺ (FIG. 24C), γδ T+/ROR-γt+(FIG. 24D) and CD8⁺/IFN-γ⁺ (FIG. 24E) and tumor mass in 8 mice per group was assessed with calipers until 10% of body mass or 14 days of growth (FIG. 24F).

### Example 12

### MDSCs Highly Express PFKFB3

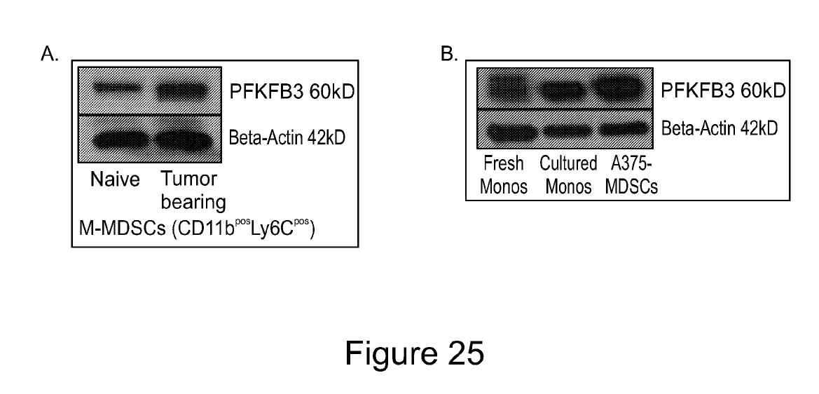

MDSCs directly suppress the activation of CD8⁺ T cells that are required for cancer immunity. Like Th17 and γδT17 cells, MDSCs have been found to over express HIF-1α and we thus postulated that MDSCs would over-express the HIF-1α target gene, PFKFB3. CD11b⁺GR-1^{dim} Ly-6G⁻ monocytic MDSCs (M-MDSC) were isolated from spleens of C57BL/6 mice bearing a s.c. B16-F10 melanoma tumor. PFKFB3 protein expression was analyzed in M-MDSC and in monocytes isolated from spleens of naïve mice. High PFKFB3 protein expression was observed in monocytic MDSCs isolated from mice (FIG. 25A) and humans (FIG. 25B).

### Example 13

### MDSCs Exposed to PFK-158 During Differentiation Caused by A375 Human Melanoma Cells have Reduced Ability to Suppress T Cell Activation

The effect of the PFKFB3 inhibitor PFK-158 was assessed on human MDSC differentiation and subsequent function using their suppressive activity against activated T cells as a measure of differentiation.

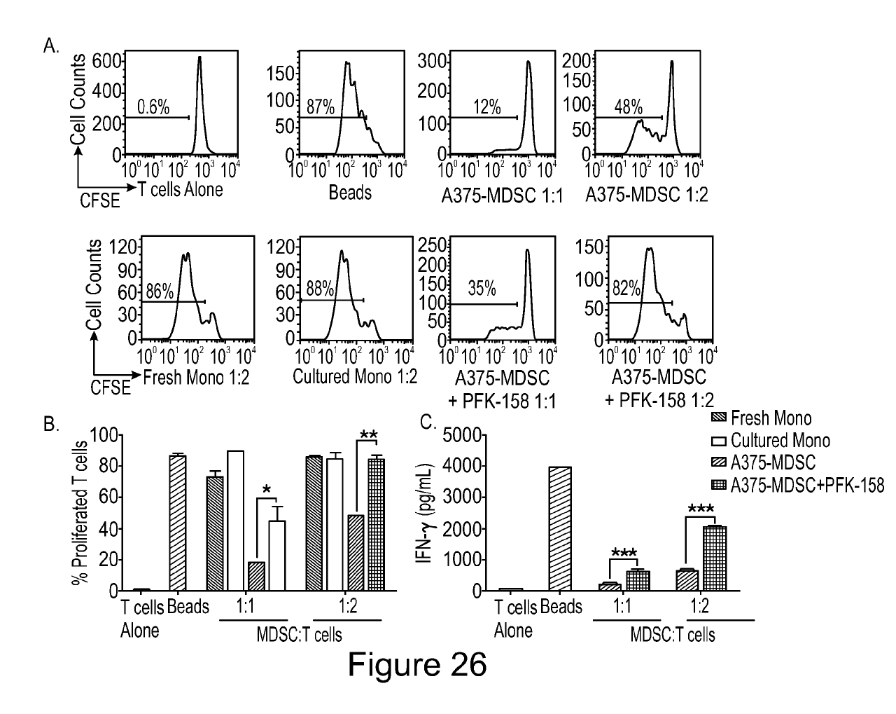

CD14⁺ cells isolated from PBMCs obtained from healthy donors were co-cultured with A375 tumor cells in a 6-well plate. Tumor/monocyte co-cultures were treated twice with 158 (5 μM on day 0 and 5 μM on day 2) or 0.1% DMSO (vehicle control). A375 co-cultured monocytes (both untreated and 158 treated) were harvested by gently scraping after 64-68 hours of culture and CD11b⁺ A375-MDSCs were purified by AutoMACS (FIG. 26 A, B). CD11b⁺ MDSCs were isolated from A375:monocyte co-cultures that were either untreated (A375-MDSC) or treated with 158 (A375-MDSC+158; 5 μM, day 0 and 5 μM, day 2). Indicated MDSCs were then added to CFSE-labeled autologous T cells at the ratios shown and cultured with anti-CD3/anti-CD28 beads for 4 days. Representative histograms indicating the percentage of proliferated T cells are shown in FIG. 26.

A marked reduction was observed in the ability of MDSCs that had been exposed to PFK-158 during differentiation caused by A375 human melanoma cells to suppress T cell activation induced by anti-CD3/anti-CD28 micro-bead co-stimulation.

### Example 14

### PFKFB3 Inhibition in Established Melanoma Cell Line-Educated MDSCs Reduces their Suppressive Function

Human MDSCs that had been permitted to differentiate in the absence of PFK-158 but were exposed to PFK-158 just prior to co-incubation with T cells were deficient in their ability to suppress T cell activation.

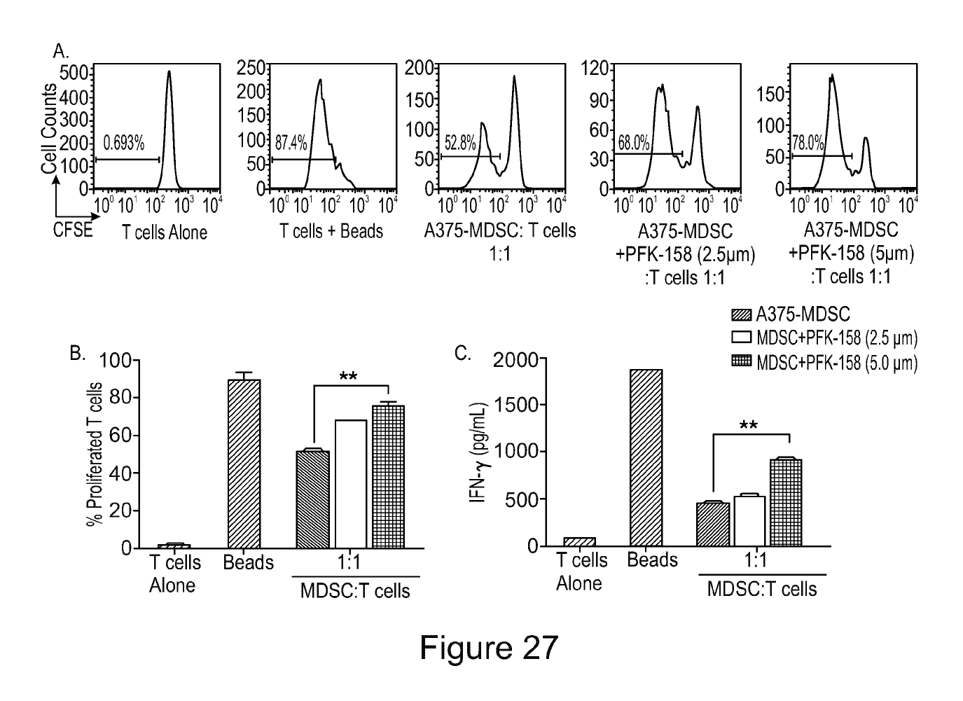

CD11b⁺ MDSCs were isolated from A375:monocyte co-cultures (A375-MDSC). A375-MDSCs were pre-treated with or without 5 μM PFK158 for 24 hours, washed extensively, and then added to CFSE-labeled autologous T cells at the indicated ratio and anti-CD3/anti-CD28 beads for 4 days. Representative histograms show the percentage of proliferated T cells. See FIG. 27.

### Example 15

### PFK-158 Suppresses the Ability of Monocytic MDSCs to Suppress Chicken Ovalbumin Antigen-Specific Splenocytes Isolated from OT-II Mice

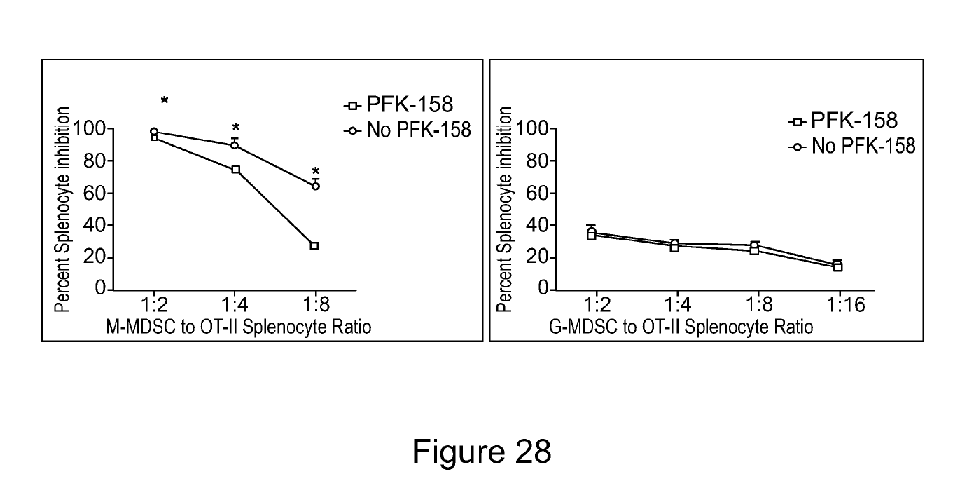

As shown in FIG. 28, antigen-specific T cell suppressive function with murine MDSCs: murine M-MDSCs (FIG. 28A) and not G-MDSCs derived from spleens of B16-F10 tumor-bearing mice (FIG. 28B) are suppressive and the antigen-specific suppressive function of M-MDSCs is reversed following ex vivo treatment with PFK-158.

Taken together, these data indicate that PFKFB3 is essential for MDSC differentiation and function. Given that MDSCs, as well as Th17 and γδT17 cells, are essential to suppress T cell immunity, we believe that these data indicate that suppression of PFKFB3 may have utility to induce cancer immunity (rather than suppress cancer immunity as was previously understood).

### Example 16

### Exemplary Formulations

### PFK-158

PFK-158 is a synthetic small molecule and the drug product is a lyophilized solution of PFK-158 at 5 mg/ml and 30% w/v Captisol® (sulfobutylether b cyclodextrin, sodium salt) in water for injection at pH 3.2. The lyophilized product is to be reconstituted with water for injection leading to a clear, transparent yellowish solution free of particles. After reconstitution, the drug product will be administered directly, without further dilution, using a rapidly running IV line.

### Ipilimumab

Ipilimumab is a recombinant, human monoclonal antibody that binds to the cytotoxic T-lymphocyte-associated antigen 4 (CTLA-4). Ipilimumab is an IgG1 kappa immunoglobulin with an approximate molecular weight of 148 kDa. Ipilimumab is produced in mammalian (Chinese hamster ovary) cell culture. Ipilimumab is provided in a sterile, preservative-free, clear to slightly opalescent, colorless to pale yellow solution for intravenous infusion, which may contain a small amount of visible translucent-to-white, amorphous ipilimumab particulates. It is supplied in single-use vials of 50 mg/10 mL and 200 mg/40 mL. Each milliliter contains 5 mg of ipilimumab and the following inactive ingredients: diethylene triamine pentaacetic acid (DTPA) (0.04 mg), mannitol (10 mg), polysorbate 80 (vegetable origin) (0.1 mg), sodium chloride (5.85 mg), tris hydrochloride (3.15 mg), and Water for Injection, USP at a pH of 7.

### Pembrolizumab

Pembrolizumab for injection is a sterile, preservative-free, white to off-white lyophilized powder in single-use vials. Each vial is reconstituted and diluted for intravenous infusion. Each 2 mL of reconstituted solution contains 50 mg of pembrolizumab and is formulated in L-histidine (3.1 mg), polysorbate 80 (0.4 mg), and sucrose (140 mg). The solution may contain hydrochloric acid/sodium hydroxide to adjust pH to 5.5.

### Nivolumab

Nivolumab is a human monoclonal antibody that blocks the interaction between PD-1 and its ligands, PD-L1 and PD-L2. Nivolumab is an IgG4 kappa immunoglobulin that has a calculated molecular mass of 146 kDa. Nivolumab solution is a sterile, preservative-free, non-pyrogenic, clear to opalescent, colorless to pale-yellow liquid that may contain light (few) particles. Nivolumab for intravenous infusion is supplied in single-dose vials. Each mL of nivolumab solution contains nivolumab 10 mg, mannitol (30 mg), pentetic acid (0.008 mg), polysorbate 80 (0.2 mg), sodium chloride (2.92 mg), sodium citrate dihydrate (5.88 mg), and Water for Injection, USP. The solution may contain hydrochloric acid and/or sodium hydroxide to adjust pH to 6.

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of treating cancer comprising administering to a subject in need thereof a synergistic, therapeutically effective amount of (1) (E)-1-(pyridyn-4-yl)-3-(7-(trifluoromethyl)quinolin-2-yl)-prop-2-en-1-one (PFK-158); and (2) an immune checkpoint inhibitor.

2. The method of claim 1, wherein the immune checkpoint inhibitor is an anti-CTLA-4 therapy selected from the group consisting of ipilimumab, tremelimumab, and combinations thereof.

3. The method of claim 2, wherein the immune checkpoint inhibitor is ipilimumab.

4. The method of claim 1, wherein the immune checkpoint inhibitor is an anti-PD-1 therapy selected from the group consisting of nivolumab, pembrolizumab, pidilizumab, MEDI0680, and combinations thereof.

5. The method of claim 1, wherein the immune checkpoint inhibitor is an anti-PD-L1 therapy selected from the group consisting of atezolizumab, BMS-936559, MEDI4736, MSB0010718C, and combinations thereof.

6. The method of claim 1, wherein the treatment is administered intravenously.

7. A method of stimulating anti-tumor immunity in a subject in need thereof comprising administering to the subject a therapeutically effective amount of (E)-1-(pyridyn-4-yl)-3-(7-(trifluoromethyl)quinolin-2-yl)-prop-2-en-1-one (PFK-158).

8. The method of claim 7, further comprising administering an effective amount of an immune checkpoint inhibitor.

9. The method of claim 8, wherein the immune checkpoint inhibitor is an anti-CTLA-4 antibody.

10. The method of claim 8, wherein the immune checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C, and combinations thereof.

11. A method of synergistically increasing the activity of an immune checkpoint inhibitor comprising administering to a subject in need thereof synergistic, therapeutically effective amount of (1) (E)-1-(pyridyn-4-yl)-3-(7-(trifluoromethyl)quinolin-2-yl)-prop-2-en-1-one (PFK-158) and (2) the immune checkpoint inhibitor.

12. The method of claim 11, wherein the immune checkpoint inhibitor is an anti-CTLA-4 therapy selected from the group consisting of ipilimumab, tremelimumab, and combinations thereof.

13. The method of claim 12, wherein the immune checkpoint inhibitor is ipilimumab.

14. The method of claim 11, wherein the immune checkpoint inhibitor is an anti-PD-1 therapy selected from the group consisting of nivolumab, pembrolizumab, pidilizumab, MEDI0680, and combinations thereof.

15. The method of claim 11, wherein the immune checkpoint inhibitor is an anti-PD-L1 therapy selected from the group consisting of atezolizumab, BMS-936559, MEDI4736, MSB0010718C, and combinations thereof.

16. A pharmaceutical composition comprising:
(a) a therapeutically effective amount of (E)-1-(pyridyn-4-yl)-3-(7-(trifluoromethyl)quinolin-2-yl)-prop-2-en-1-one (PFK-158);
(b) a therapeutically effective amount of at least one immune checkpoint inhibitor; and
(c) at least one pharmaceutically-acceptable carrier.

17. The pharmaceutical composition of claim 16, wherein the immune checkpoint inhibitor is selected from the group consisting of ipilimumab, tremelimumab, nivolumab, pembrolizumab, pidilizumab, MEDI0680, atezolizumab, BMS-936559, MEDI4736, MSB0010718C, and combinations thereof.

18. The pharmaceutical composition of claim 16, wherein the immune checkpoint inhibitor is an therapy selected from the group consisting of anti-CTLA-4, anti-PD-1, anti-PD-L1, and combinations thereof.

19. A method of immunotherapy comprising administering to a subject in need thereof a therapeutically effective amount of (E)-1-(pyridyn-4-yl)-3-(7-(trifluoromethyl)quinolin-2-yl)-prop-2-en-1-one (PFK-158).

20. The method of claim 19, wherein the immunotherapy treats an autoimmune condition selected from the group consisting of lupus, rheumatoid arthritis, multiple sclerosis, ulcerative colitis, inflammatory bowel disease, asthma, Crohn's disease, psoriasis, and diabetes mellitus type 1.
